(19) United States
(12) Patent Application Publication
(10) Pub. No.: US 2005/0043534 A1
(43) Pub. Date: Feb. 24, 2005

(54) Modulators of ceramidase and methods of used based thereon

(76) Inventors: Alicja Bielawska, Charleston, SC (US); Yusuf A. Hannun, Sullivan's Island, SC (US); Zdzislaw Szulc, Charleston, SC (US); Julnar Usta, Charleston, SC (US); Samer El Bawab, Charleston, SC (US)

Correspondence Address: JONES DAY, 222 EAST 41ST ST, NEW YORK, NY, 10017, US

(21) Appl. No.: 10/483,618

(22) PCT Filed: Jul. 11, 2002
(86) PCT No.: PCT/US02/22151

Related U.S. Application Data
(60) Provisional application No. 60/304,710, filed on Jul. 11, 2001.

(51) Int. Cl. C07C 231/02; C07D 207/46; C07D 209/18

(52) U.S. Cl. 546/102; 546/176; 546/337; 548/495; 548/571; 554/51

(57) ABSTRACT

The present invention relates to compounds which can be used as inhibitors of mitochondrial ceramidase, in particular human mitochondrial ceramidase. The invention also relates to methods of designing and making the compounds, as well as methods screening for compounds that inhibit mitochondrial ceramidase. The invention also relates to the use of the compounds as a regulator of the level of ceramide by inhibiting ceramidase activity. The invention also relates to methods for the prevention and treatment of diseases associated with cell overproliferation and sphingolipid signal transduction including cancer, cardiovascular diseases, and inflammation.

This application claims priority of U.S. Provisional Patent Application No. 60/304,710 filed Jul. 11, 2001, which is incorporated by reference herein in its entirety.

### 1. FIELD OF THE INVENTION

The present invention relates to compounds which can be used as modulators of mitochondrial ceramidase, in particular human mitochondrial ceramidase. The invention further relates to pharmaceutical compositions comprising at least one such compound. The invention also relates to methods of making the compounds, methods of designing, and method of screening the compounds that inhibit mitochondrial ceramidase. The invention also relates to methods for the prevention and treatment of diseases associated with cell overproliferation and sphingolipid signal transduction. In particular, the invention relates to the use of the compounds as inhibitors in the regulation of the level of ceramide by inhibiting ceramidase activity.

### 2. BACKGROUND

Ceramide is a potent signal transducer that affects cell growth, differentiation and death (Hannun, Y. A. (1996) Science 274, 1855-1859; Obeid, L. M., Linardic, C. M., Karolak, L. A., and Hannun, Y. A. (1993) Science 259, 1769-1771; Perry, D. K. and Hannun, Y. A., (1998) Biochim Biophys Acta 436, 233-243). It occupies a central position in sphingolipid metabolism. As an acceptor of carbohydrates, phosphorylcholine and phosphate, it serves as precursor of the various complex sphingolipids. Alternatively, the enzymatic breakdown of these sphingolipids releases ceramide which may consequently be hydrolyzed into fatty acid and sphingosine; the latter exerting bioeffector functions on its own as well as acting as a precursor of sphingosine phosphate, another signal mediator and regulator of various cell functions. A controlled level of ceramide, therefore, reflects an intricate balance between the catabolic and anabolic pathways of ceramide.

Multiple enzymes are directly involved in regulating intracellular ceramide concentration. These include ceramide-generating enzymes such as ceramide synthase, cerebrosidase, sphingomyelinase and ceramide-consuming enzymes such as cerebroside synthase, sphingomyelin synthase, ceramide kinase and ceramidase (Luberto, C. and Hannun, Y. A., (1999) Lipids 34, 5-11).

Ceramidases are enzymes that hydrolyze ceramides at the amide bond linking the sphingosine moiety to the fatty acids. In that sense they provide a target site for regulating ceramide-sphingosine inter-conversion (Hassler, D. F. and Bell, R. M., (1993) Adv. Lipid Res. 49-57). At least three different types of ceramidases have been reported. A lysosomal acid ceramidase, the defect of which underlies the human disorder Farber's disease (Sugita, M., Dulaney, J. T. and Moser, H. W., (1972) Science 178, 1100-1102), was purified from human urine (Koch, J., Gartner, S. Li. C-M., Quinten, L. E., Bernardo, K., Levarn, O., Schnabel, D., Desnick, R. J., Schuchman, E. H., and Sandhoff, K., (1996) J. Biol. Chem. 271, 33110-33115), and the cDNA encoding the enzyme was also cloned from mouse brain and human fibroblasts (Koch, J., Gartner, S. Li. C-M., Quinten, L. E., Bernardo, K., Levarn, O., Schnabel, D., Desnick, R. J., Schuchman, E. H., and Sandhoff, K., (1996) J. Biol. Chem. 271, 33110-33115; Li, C.-M., Hong, S. B., Kopal, G., He, X., Linke, T., Hou, W. S., Koch, J., Gatt, S., Sandhoff, K., and Shuchman, E. H., (1998) Genomics 50, 267-274). Alkaline ceramidases, CDase-I and CDase-II, were purified from guinea pig skin (Yada, Y., Higuch, K., and Imokawa, G., (1995) J. Biol. Chem. 270, 12677-12684) and from gram-negative bacterium Pseudomonas aeruginosa (Okino, N., Tani, M., Imayama, S., and Ito, M., (1998) J. Biol. Chem. 273, 14368-14373; Okino, N., Ichinose, S., Omori, A., Imayama, S., Nakamura, T., and Ito, M., (1999) J. Biol. Chem. 274, 36616-36622).

A non-lysosomal, ceramidase with a neutral to alkaline pH optimum was also purified to homogeneity from rat brain (El-Bawab, S., Bielawska, A., and Hannun, Y. A., (1999) J. Biol. Chem. 274, 27948-27955) and cloned from mouse (Tani, M., Okino, N., Mori, K., Tanigawa, T., Izu, H., and Ito, M., (2000) J. Biol. Chem. 235, 11229-11234) and human (El-Bawab, S., Roddy, P., Qian, T., Bielawska, A., Lemasters, J. J., and Hannun, Y. A., (2000) J. Biol. Chem. 275, 21508-21513). The human form was found to localize to mitochondria (El-Bawab, S., Roddy, P., Qian, T., Bielawska, A., Lemasters, J. J., and Hannun, Y. A., (2000) J. Biol. Chem. 275, 21508-21513).

Citation of references hereinabove shall not be construed as an admission that such references are prior art to the present invention.

### 3. SUMMARY OF THE INVENTION

The present invention provides mitochondrial ceramidase inhibitors. The invention is based on the observation that the interaction of mitochondrial ceramidase with its ligand occurs in a high affinity-low specificity manner, which is in contrast to catalysis which is highly specific for D-erythro-ceramide (D-e-Cer) but occurs with a lower affinity.

The compounds of the invention are structurally related to of ceramide or sphingosine. In one embodiment of the invention, the compounds of the invention are designed according to modifications of key structural elements in ceramide and sphingosine, including stereochemistry, the primary and secondary hydroxyl groups, the trans double bond in the sphingosine backbone, and the amide bond. In general, the compounds of the invention interfere with one or more of the following structure of the ceramide or sphingosine: the primary and secondary hydroxyl groups, the C4-C5 double bond, the trans configuration of the C4-C5 double bond, or the NH-protons from either the amide of ceramide or the amine of sphingosine.

In specific embodiments, the mitochondrial ceramidase inhibitors of the invention are 1) all stereoisomers of D-erythro-ceramide (D-e-Cer) with an IC₅₀ (the concentration of an inhibitor at which ceramidase activity is inhibited by 50% of the level observed in the absence of the inhibitor) of the range 0.01-0.8 mole %, preferably an IC₅₀ of 0.11, 0.21 and 0.26 mole %, for the L-threo, D-threo and L-erythro isomers respectively; 2) all stereoisomers of sphingosine with IC₅₀ ranging from 0.01-0.8 mole %, preferably an IC₅₀ of 0.04 to 0.14 mole %, for the N-alkyl-D-erythro-sphingosine (most preferably for N-Me-Sph, IC₅₀ 0.13 mole %); and 3) D-erythro-urea-C₁₆-ceramide (most preferably for C₁₆-urea-Cer IC₅₀ 0.33 mole %). In preferred embodiments, the compounds are potent competitive inhibitors: urea-ceramide (C₁₆-urea-Cer) and ceramine (C₁₈-ceramine).

In various preferred embodiments, the invention encompasses potent mitochondrial ceramidase inhibitors, such as but not limited to, D-erythro-sphinganine with an IC₅₀ of the range 0.01-0.8 mole % (more preferably for D-e-dh-Sph, IC₅₀ 0.20 mole %), D-erythro-dehydro sphingosine with an IC₅₀ of the range 0.01-0.8 mole % (more preferably for D-e-deh-Sph, IC₅₀ 0.25 mole %), (2S)-3-keto-sphinganine with an IC₅₀ of the range 0.01-0.8 mole % (more preferably for 3-keto-dh-Sph, IC₅₀ 0.34 mole %), (2S) 3-keto-ceramide with an IC₅₀ of the range 0.01-0.8 mole % (more preferably for 3-keto-C₁₆-Cer, IC₅₀ 0.60 mole %).

In other embodiments, the invention encompasses weaker mitochondrial ceramidase inhibitors, such as but not limited to, 1-O-Methyl-D-erythro-sphingosine (1-O-Me-Sph), cis-D-erythro-sphingosine (cis-D-e-Sph), (2S)-3-keto-sphingosine (3-keto-Sph), (2S)-3-keto-dehyrosphingosine (3-keto-deh-Sph), and N,N-dimethyl-D-erythro-sphingosine (N,N-diMe-Sph).

In yet another embodiment, the invention provides the use of ceramide-1-phosphate (Cer-1-P) and sphingosine-1-phosphate (Sph-1-P) to stimulate mitochondrial ceramidase.

The present invention provides method of designing and screening for compounds that inhibit mitochondrial ceramidase. Methods of making the compounds that inhibits mitochondrial ceramidase are also provided.

The present invention encompasses methods, pharmaceutical compositions, and dosage forms for the treatment and prevention of disorders that are ameliorated by the inhibition of mitochondrial ceramidase in mammals, including humans. Examples of such disorders include, but are not limited to, various cancers, hyperproliferative diseases, cardiovascular diseases, and inflammation. The methods of the invention comprise administering to a patient in need of such treatment or prevention a therapeutically or prophylactically effective amount of a compound of the invention, or a pharmaceutically acceptable salt,-or solvate thereof.

Pharmaceutical compositions of the invention comprise a therapeutically or prophylactically effective amount of a mitochondrial ceramidase inhibitor. Preferred compounds are those that are active in decreasing cell survival and viability (e.g., which can be demonstrated in in vitro assays or in breast cancer cell line assays, or can be identified using in vitro assays, animal models, or cell culture assays). Pharmaceutical compositions of the invention can further comprise other anticancer or anti-inflammatory drug substances.

The present invention provides compounds for increasing mitochondrial ceramidase activity having the Formula V and VI as shown below. In a more preferred embodiment, such compounds include ceramide 1-phosphate and sphingosine 1-phosphate.

The invention also provides methods of treatment of disorders involving deficient cell proliferation or growth, or in which cell proliferation is otherwise desired (e.g., degenerative disorders, growth deficiencies, lesions, physical trauma) by administering compounds that activates mitochondrial ceramidase (e.g., ceramide-1-phosphate (Cer-1-P) and sphingosine-1-phosphate (Sph-1-P). Activating ceramide function can also be done to grow larger animals and plants, e.g., those used as food or material sources.

The present invention further relates to a method of synthesizing cis-D-erythro-sphingosine, which comprises regioselective catalytic hydrogenation of N-Boc-4.5-dehydro-D-erythro-sphingosine using Raney® 2800 nickel catalyst performed in ethanol solution, in the presence of pyridine; deprotecting of the formed 9:1 mixture of cis/trans isomers of N-Boc-D-erythro-sphingosine using chlorotrimethylsilane in methanol, and final separation of the formed cis/trans-D-erythro-sphingoid bases using silica gel column chromatography and chloroform-methano1-concentrated ammonium hydroxide (5:1:0.05 v/v/v/) as an eluent system.

The present invention also further relates to a method of synthesizing of varied chains urea analogs of ceramides, which comprises regioselective condensation of sphingosine base with an alkyl isocyanate, performed in an inert solvent system at room temperature.

### 3.1 Abbreviations

A number of abbreviations are used throughout as detailed below:

Mt-CDase, mitochondrial ceramidase; D-erythro-C₁₈-ceramide, D-e-C₁₈-Cer; L-erythro-C₁₈-ceramide, L-e-C₁₈-Cer; L-threo-C₁₈-ceramide, L-t-C₁₈-Cer; D-threo-C₁₈-ceramide, D-t-C₁₈-Cer; cis-D-erythro-C₁₆-ceramide, cis D-e-C₁₆-Cer; 1-O-methyl-D-erythro-C₁₆-ceramide, 1-O-Me-C₁₆-Cer; 3-O-methyl-D-erythro-C₁₆-ceramide, 3-O-Me-C₁₆-Cer; 3-keto-C₁₆-ceramide, 3-keto-C₁₆-Cer; D-erythro-C₁₆-ceramide-1-phosphate, Cer-1-P; D-erythro-C₁₆-urea-ceramide, C₁₆-urea-Cer, N-methyl-D-erythro-C₁₆-ceramide, N-Me-C₁₆-Cer; D-erythro-sphingosine, D-e-Sph; L-erythro-sphingosine, L-e-Sph, L-threo-sphingosine, L-t-Sph; D-threo-sphingosine, D-t-Sph; cis-D-erythro-sphingosine, cis D-e-Sph; D-erythro-dihysphingosine, D-e-dh-Sph; D-erythro-dehydrosphingosine, D-e-deh-Sph; 1-O-methyl-D-erythro-sphingosine, 1-O-Me-Sph; 3-O-methyl-D-erythro-sphingosine, 3-O-Me-Sph; (2S)-3-keto-sphingosine, 3-keto-Sph; (2S)-3-keto-dihydrosphingosine, 3-keto-dh-Sph; (2S)-3-keto-dehydrosphingosine, 3-keto-deh-Sph; D-erythro-sphingosine-1-phosphate, Sph-1-P; N-methyl-D-erythro-sphingosine, N-Me-Sph; N-stearyl-D-erythro-sphingosine, C₁₈-ceramine; N,N-dimethyl-D-erythro-sphingosine, N,N-diMe-Sph.

### 4. BRIEF DESCRIPTION OF THE FIGURES

FIG. 1. Scheme of structural modifications of ceramides and sphingosines.

FIG. 2. Effect of ceramide stereoisomers on mitochondrial ceramidase activity. a) Mitochondrial ceramidase activity was determined using the HPLC assay for sphingoid products as described under “Experimental Procedures” at constant D-erythro-C₁₈-ceramide (0.625 mole %) while varying the concentration of ceramide stereoisomers. Results are means of 2 separate experiments. b) Double reciprocal representation of L-erythro-C₁₈-ceramide at 0.125 and 0.314 mole %. c) Effect of cis D-erythro-ceramide on mt-CDase activity. Results are average ±SD of 3 different determinations.

FIG. 3. Effects of functional group modification of ceramide on mitochondrial ceramidase. Mitochondrial ceramidase activity was carried out as described under “Experimental Procedure” at constant [³H]-D-erythro-C₁₆-ceramide while varying the concentrations of: a) Cer-1-P; and b) 1-O-methylceramide (1-O-Me-C₁₆-Cer), 3-O-methyl-ceramide (3-O-Me-C₁₆-Cer), N-methyl-ceramide (N-Me-C₁₆-Cer), and 3-keto-ceramide (3-keto-C₁₆-Cer). Results are means ±SD of three separate experiments.

FIG. 4. The effects of products of the ceramidase reaction on mitochondrial ceramidase activity. The effects of increasing concentrations of palmitate a) and sphingosine stereoisomers b) on mitochondrial ceramidase activity were determined using radiolabeled ceramide substrate. c) The effects of desaturation, saturation, and configuration of the 4-5 double bond on ceramidase activity. Enzyme activity was determined at constant D-erythro-ceramide while varying the concentration of D-erythro-sphingosine; D-erythro-dihydrosphingosine; D-erythro-dehydrosphingosine; and cis D-erythro-sphingosine. Results are means ±SD of three separate experiments.

FIG. 5. Effects of the modified hydroxyl groups of sphingosine on mitochondrial ceramidase. Mitochondrial ceramidase activity was carried out as described in Section 6 at constant D-erythro-ceramide while varying the concentrations of a) Sph-1-P; b) D-e-Sph, 1-O-Me-Sph, 3-O-Me-Sph; or c) 3-keto-Sph, 3-keto-dh-Sph, and 3-keto-deh-Sph. Results are expressed as means ±SD of three separate experiments.

FIG. 6. The effects of N-alkyl-sphingosines on mitochondrial ceramidase. Mitochondrial ceramidase was determined as described in Section 6 at constant D-erythro-ceramide while varying the concentration of a) D-e-Sph (re-plotted from FIG. 4b for comparison), N-Me-Sph, and N,N-diMe-Sph. b) dose response effect of ceramine on mt-CASE activity. c) Double reciprocal representation of ceramine effect at 0.125 and 0.314 mole %. Results are expressed as means ±SD of three separate experiments for (a) and (b), and as means of two different experiments for (c).

FIG. 7. The effects of the ceramide-urea isoster on mitochondrial ceramidase. Mitochondrial ceramidase activity was determined using the radio labeled substrate. a) Inhibition of mitochondrial ceramidase by urea-C₁₆-ceramide. b) double reciprocal representation of C-₁₆-urea-CER effect at 0.125 and 0.251 mole %. c) effects of increasing concentration of alkyl amines: C₈, C₁₂, and C₁₈ on mitochondrial ceramidase. Results are means ±SD of three separate experiments for (a), and means of two different experiments for (b) and (c).

FIG. 8. Chemical formula of representative compounds for the inhibition of mitochondrial ceramidase.

FIG. 9: Effects of Urea-C16-Ceramide on MCF-7 and HEK-293 cells. Cell viability was measured by MTT assay. Treatments were for about 18 hours.

### 5. DETAILED DESCRIPTION OF THE INVENTION

The present invention relates to compounds useful for the modulation of mitochondrial ceramidase. The present invention also includes methods of designing, methods of making, and methods of screening for compounds that inhibit or increase mitochondrial ceramidase. As used herein, the term “mitochondrial ceramidase” refers to the cereamidase enzymes as described in PCT publication WO 01/55410, which is incorporated herein by reference in its entirety. Preferably, the mitochondrial ceramidase is a mammalian mitochondrial ceramidase, such as but not limited to those ceramidase described in El Bawab et al. (1999, J. Biol. Chem. 274, 27948-27955), Tani et al., (2000, J. Biol. Chem. 235, 11229-11234) and mitochondrial ceramidase of other species that can be purified by the methods described in El Bawab et al. (1999). Most preferably, the mitochondrial ceramidase is human mitochondrial ceramidase (El-Bawab et al., 2000, J. Biol. Chem. 275, 21508-21513).

The present invention also includes methods and compositions for the prevention and treatment of diseases associated with cell overproliferation and sphingolipid signal transduction. 5.1 Modulators of Ceramidase

The regulation of ceramide and sphingosine levels in vivo is achieved by many enzymes, particularly ceramidases. A non-lysosomal ceramidase was isolated and characterized from rat brain (El-Bawab, S., Bielawska, A., and Hannun, Y. A., (1999) J. Biol. Chem. 274, 27948-27955). The human form was found to localize to mitochondria (El-Bawab, S., Roddy, P., Qian, T., Bielawska, A., Lemasters, J. J., and Hannun, Y. A., (2000) J. Biol. Chem. 275, 21508-21513). A better understanding of the-role and mechanism of this mitochondria-associated ceramidase (mt-CDase) is needed.

The present inventors discovered that this mt-CDase specifically hydrolyzes the D-erythro-isomer of ceramide. These findings indicate that the mt-CDase harbors a very specific recognition site for its substrate, which renders it a ceramidase but not a generalized amidase. This high specificity also suggests that the enzyme plays a specific role in the regulation of endogenous levels of ceramide and sphingosine.

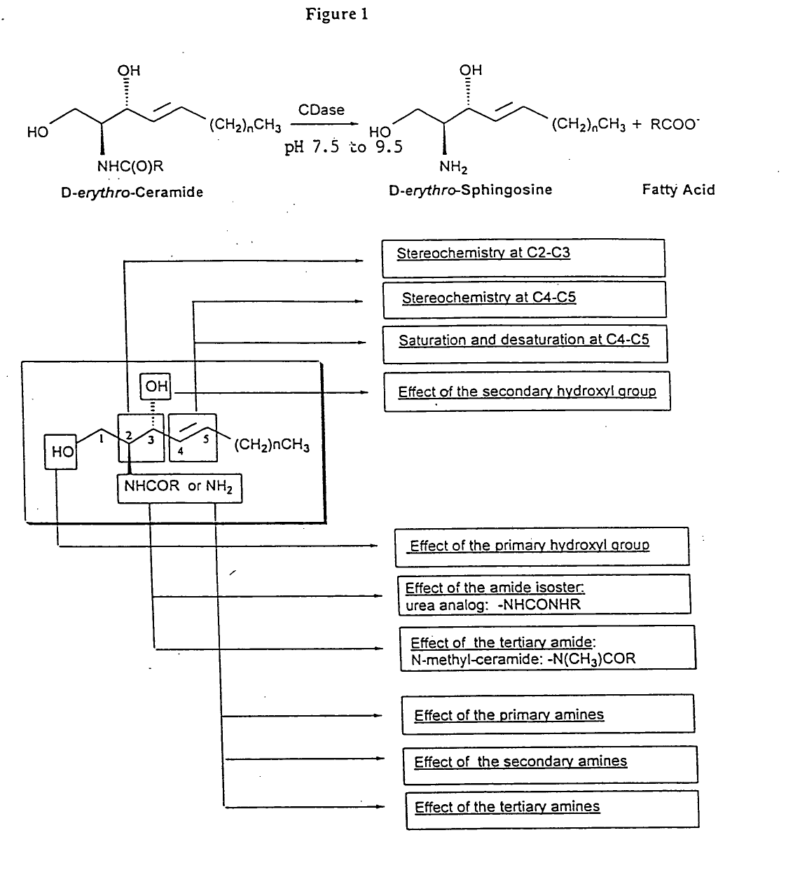

The present inventors also discovered key features of the ceramide structure necessary for recognition by and interaction with mitochondrial ceramidase. Specifically, the present inventors discovered that the naturally-occurring D-erythro-ceramide, [N-acyl-(2S,3R,4E)-2-amino-1,3-dihydroxy-octadecene-4]exhibits several important features including the presence of the 1 and 3 hydroxyl groups, two chiral centers and at least two distinct elements of rigidity: the (4E) trans-alkenyl and the amide bond linkage to the (2S, 3R) chiral backbone of sphingosine (FIG. 1). These structural features are decisive elements in determining the proper conformation assumed by the substrate D-erythro-ceramide and its ability to interact effectively with the enzyme. Therefore, variation of these structural features result in analogues of ceramide and sphingosine that are candidates for inhibitors on mitochondrial ceramidase.

Analogues of ceramide were synthesized and tested for their effects on mitochondrial ceramidase. In addition, analogues of sphingosine were also developed since sphingosine is a defining component of ceramide, a product of the reaction, and a competitive inhibitor of ceramidase (El-Bawab, S., Bielawska, A., and Hannun, Y. A., (1999) J. Biol. Chem. 274, 27948-27955). The design of compounds was based mainly on investigation of 1) the stereochemistry at C2 and C3 positions; 2) the primary and secondary hydroxyl groups; 3) the 4-5 trans double bond; 4) the amide bond; and 5) the NH₂ function of sphingosine.

The present inventors discovered that many of the analogues of ceramide or sphingosine inhibit the enzyme, demonstrating that the enzyme recognizes these structures as ligands but not as substrates. The present inventors discovered that even small modifications of ceramide can either prevent interaction with the enzyme or convert the substrate into an inhibitor. These results provide significant insight into the molecular interactions of substrate (and product) with the enzyme.

Exemplary compounds of the present invention are urea-ceramide and ceramine, which are structurally highly analogous to ceramide and sphingosine. They competitively inhibit the enzyme. The modifications that are used in the method of the present invention generate a new class of inhibitors. The most preferred compound in the present invention are the unnatural optical isomers of D-erythro-ceramide and sphingosine which are highly potent inhibitors of mitochondrial ceramidase. Thus, these compounds are templates for further development of other inhibitors of mitochondrial ceramidase.

Indeed, the determination of requirements for hydrolysis of ceramide by the enzyme, shows that of all four optical isomers, only the D-erythro-ceramide is a substrate. Also, with the exception of urea ceramide analogues, the enzyme requires the amide bond bearing a long fatty acid, and the free primary and secondary hydroxyls. The enzyme also shows significant preference for the 4-5 trans double bond of the sphingoid backbone. Indeed, the enzyme does not tolerate many of the possible modifications in the ceramide structure.

As demonstrated by the examples provided hereinbelow. A few structural modifications resulted in a significant loss of affinity of enzyme to D-erythro-ceramide as judged by loss of inhibition. Neither the N-Me-ceramide, the 1 -O-Me-ceramide, the 3-O-Me-ceramide, the cis-D-e-ceramide, nor the ceramide-1-phosphate exhibited any inhibitory effects, demonstrating that these analogues do not interact with the enzyme. The lack of inhibition for these O-and N-substituted analogs indicates an important role of the free hydrogens in the functional groups of ceramide (relative interactions of the functional groups or interaction of a specific group with the enzyme) or can be related to steric effects that prevent or hinder the interaction of the analogs with the larger substituents with the enzyme.

In addition, methylation of the primary and secondary hydroxyl groups of sphingosine, reduced severely the potency of sphingosine, indicates a critical role for the primary and the secondary hydroxyl groups in interaction and recognition.

Possible role of the 3 hydroxy group in interacting with the enzyme is based on the observation that 3-keto-sphingoid bases (2S) 3-keto-Sph displayed significant loss of inhibition when compared to D-e-Sph. On the other hand, 3-keto-dh-Sph was a potent inhibitor whereas 3-keto-deh-Sph was not. The keto-group of 3-keto-dh-Sph has a higher electron density compared to either 3-keto-Sph or to 3-keto-deh-Sph as a result of the lack of conjugation with the electron deficient system (double or triple bond, respectively). This facilitates formation of a stronger hydrogen acceptor bond between the carbonyl group in 3-keto-dihydrosphingosine and ceramidase. Taken together, the hydrogen-donor properties of the 3-OH are important for the interaction of the enzyme with sphingosines.

The present inventors also discover the role of the trans 4-5 double bond in the sphingosine backbone. The cis D-erythro-ceramide did not inhibit the enzyme, and thus did not demonstrate any interaction. Similarly, the 4, 5 cis isomer of sphingosine was a weak inhibitor. Therefore the enzyme recognizes specifically the trans orientation. Since the cis bond introduces a kink in the hydrophobic chain, this creates a steric effect, preventing ceramide (and sphingosine) from fitting into the catalytic site. Moreover, reduction of the C4-C5 double bond in ceramide produces dihydroceramide, which displayed significant loss of activity as a substrate. Similarly, reduction of this bond in sphingosine reduced the extent of inhibition and increased the IC₅₀ from 0.04 mole % to 0.34 mole % for D erythro-dihydrosphingosine when compared to D-erythro-sphingosine. Therefore, the enzyme shows preferential requirement for the trans-double bond which is a component of a rigid intramolecular allylic system that may facilitate interaction with the enzyme. Hence, the presence of the trans- double bond, though not necessary, increases significantly the extent of inhibition of ceramidase.

Introduction of a methyl group into the secondary amido-function of D-erythro-ceramide (N-Me-Cer) also resulted in loss of the interaction of ceramide with the enzyme, and N-alkylation of sphingosine (N-methyl- and N-stearyl-homologs) also attenuated inhibition by sphingosine. However, N,N-dimethylation of sphingosine resulted in profound loss of activity. Since N-Me-Cer and N,N-diMe-Sph do not have the proton donor activities of the amido and amino groups of ceramide and sphingosine, respectively, these findings indicate that the NH protons contribute significantly to the interaction of ceramide and sphingosine with the enzyme. These observations suggest an important role of the free amino hydrogen of sphingosine in the interaction with the enzyme, but also can raise the issue of steric effects of the larger methyl groups.

The inventors discovered that there were several modifications that generate potent inhibitors of the enzyme, which include: 1) amide group modification into the urea analogue (IC₅₀: 0.33 mole %) and 2) chiral modifications at the C2 and C3 positions. Since all these modifications were inhibitory, they did not disrupt critical features required for interaction with the enzyme.

The present inventors observed that urea ceramide was a potent inhibitor of the enzyme, though not quite as potent as the stereoisomers of ceramide. The diminished effectiveness is related to the lower polarity of the carbonyl group in the urea moiety as the result of the extended delocalization of the electron density of the carbonyl group over the two neighboring nitrogen atoms. This factor can diminish effectiveness of the hydrogen-acceptor properties of the carbonyl group in the formation of a lipid-enzyme complex.

The observation that all stereoisomers of ceramide (and sphingosine) interact with the enzyme, with only the natural D-erythro-ceramide functioning as a substrate indicates that D-erythro-ceramide retains a unique configuration required for catalysis but not for binding- As with ceramide isomers, stereochemistry had no impact on the ability of sphingosine to inhibit the enzyme, indicating that this interaction is not stereospecific. Such stereochemical interactions whereby one enantiomer is a substrate and the other is an inhibitor is uncommon, even though enantiomeric selectivity is the rule in enzyme-mediated catalysis. Recently, such interaction is seen in diastereoisomers of a-D-mannosyl transferase substrates with enantiomeric configuration at the polar head group (phosphoinositols). This ability of the enzyme to recognize ceramide isomers and analogues as ligands (inhibitors) but not substrates signal the mechanisms of interaction and on the respective structural requirements.

Modifications that allow hydrolysis include: i) variations in the chain length of the fatty acids (C₁₂-C₂₄); ii) replacement of saturated fatty acids with mono-unsaturated fatty acids; the latter were shown to exhibit a higher affinity to mitochondrial ceramidase than their saturated counterparts; iii) ceramides with a shorter sphingosine (C₁₀); and iv) oxidation of the 3-hydroxyl group into the 3-keto-ceramide yielded a competitive substrate.

In one embodiment, the present invention provides the uses of compounds for modulation of mitochondrial ceramidase having Formula I: 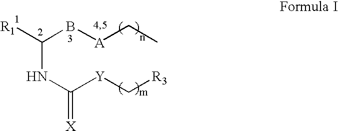
wherein
A is —CH₂CH₂—, trans —CH═CH—, or —C≡C—;
B is —CH₂—, —CH(OH)—, —CH(alkyl, —C(═O)—, —C(═NOH), or —C(═N—NH₂)—;
X is O, S, or NH;
Y is NH, O, C═O, CHR₄, CH₂C═O, or CH₂CHR₄;
R₁ is CH₃, CH₂OH, CH₂SH, CH₂—NH₂, CH₂N₃, CH₂—NH—OH, CH═N—OH, CH═N—NH₂, C(═O)H, C(═O)CH₃, C(═O)CF₃, C(═O)NH₂, CH₂R₅, C(OH)R₅, or C(═O)R₅;
R₃ is H, CH₃, OH, NH₂, NH₂.HCl, NHC(═O)NH(CH₂)_{L}N(CH₃)₂, NHCONH(CH₂)_{L}N(CH₃)₂.HCl or C(═O)R₅;
R₄ is H, OH, OR₅, or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle (e.g., imidazolyl, tetrazolyl, pyridyl, pyrimidyl, pyrazinyl, triazolyl, morpholinyl, benzoxazyl, benzofurazyl, oxazolopyridinyl); R₆-phenyl; or R₆-condensed aromatic ring system (e.g., indenyl, naphthyl, phenanthyl, anthracenyl, fluorenyl);
R₆ is H, Cl, OH, CH₃, NO₂, NH₂, N(CH₃)₂, or NHC(═O)(CH₂)ₖCH₃;
L is an integer from 2 to10 ;
k is an integer from 5 to16;
n is an integer from 4 to 20; and
m is an integer from 0 to 26;
and wherein the compound is not (2S, 3R, 4E) ceramide (R₁ is CH₂OH; B is —CH(OH)—; X is O; Y is CHR₄; A is trans —CH═CH—; R₃ is CH₃, H, or OH; R₄ is H or OH; n is an integer from 12 to16; and m is an integer from 0 to 24).

In another embodiment, the present invention provides the uses of compounds for modulation of mitochondrial ceramidase having Formula II: 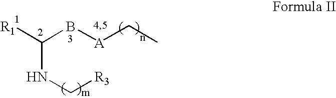
wherein
A is —CH₂CH₂—, trans —CH═CH—, or —C≡C—;
B is —CH₂—, —CH(OH)—, —CH(alkyl)—, —C(═O)—, —C(═NOH)—, or —C(═N—NH₂)—;
R₁ is CH₃, CH₂OH, CH₂SH, CH₂—NH₂, CH₂N₃, CH₂—NH—OH, CH═N—OH, CH═N—NH₂, C(═O)H, C(═O)CH₃, C(═O)CF₃, C(═O)NH₂, CH₂R₅, C(OH)R₅, or C(═O)R₅;
R₃ is H, CH₃, OH, NH₂, NH₂.HCl, NHC(═O)NH(CH₂)_{L}N(CH₃)₂, NHCONH(CH₂)_{L}N(CH₃)₂.HCl or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle (e.g., imidazolyl, tetrazolyl, pyridyl, pyrimidyl, pyrazinyl, triazolyl, morpholinyl, benzoxazyl, benzofurazyl, oxazolopyridinyl); R₆-phenyl; or R₆-condensed aromatic ring system (e.g., indenyl, naphthyl, phenanthyl, anthracenyl, fluorenyl);
R₆ is H, Cl, OH, CH₃, NO₂, NH₂, N(CH₃)₂, or NHC(═O)(CH₂)ₖCH₃;
L is an integer from 2 to10;
k is an integer from 5 to16;
n is an integer from 4 to 20; and
m is an integer from 0 to 26.

In yet another embodiment, the present invention provides compounds for modulation of mitochondrial ceramidase having Formula III: 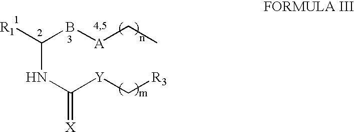
wherein
A is —CH₂CH₂—, trans —CH═CH—, or —C≡C—;
B is —CH₂—, —CH(OH)—, —CH(alkyl)—, —C(═O)—, —C(═NOH)—, or —C(═N—NH₂)—;
X is O, S, or NH;
Y is NH, O, C═O, CHR₄, CH₂C═O, or CH₂CHR₄;
R₁ is CH₃, CH₂OH, CH₂SH, CH₂—NH₂, CH₂N₃, CH₂—NH—OH, CH═N—OH, CH═N—NH₂, C(═O)H, C(═O)CH₃, C(═O)CF₃, C(═O)NH₂, CH₂R₅, C(OH)R₅, or C(═O)R₅,
R₃ is H, CH₃, OH, NH₂, NH₂.HCl, NHC(═O)NH(CH₂)_{L}N(CH₃)₂, NHCONH(CH₂)_{L}N(CH₃)₂.HCl or C(═O)R₅;
R₄ is H, OH, OR₅, or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle (e.g., imidazolyl, tetrazolyl, pyridyl, pyrimidyl, pyrazinyl, triazolyl, morpholinyl, benzoxazyl, benzofurazyl, oxazolopyridinyl); R₆-phenyl; or R₆-condensed aromatic ring system (e.g., indenyl, naphthyl, phenanthyl, anthracenyl, fluorenyl);
R₆ is H, Cl, OH, CH₃, NO₂, NH₂, N(CH₃)₂, or NHC(═O)(CH₂)ₖCH₃;
L is an integer from 2 to10;
k is an integer from 5 to16;
n is an integer from 4 to 20;
m is an integer from 0 to 26;
with the proviso that the compound is not (2S, 3R, 4E), (2S, 3R, 4E), (2R, 3R, 4E), or (2R, 3R, 4E) ceramide wherein R₁ is CH₂OH; B is —CH(OH)—; X is O; Y is CHR₄; A is trans —CH═CH—; R₃ is CH₃, H, or OH; R₄ is H or OH; n is 12; and m is an integer from 0 to 24; and
and wherein the compound is not (2S, 3R, 4E) 3-keto ceramide (R₁ is CH₂OH; B is —C(═O); X is O; Y is CHR₄; A is trans —CH═CH—; R₃ is CH₃ or OH; R₄ is H or OH; n is 12; and m is 13).

In yet another embodiment, the present invention provides compounds for modulation of mitochondrial ceramidase having Formula IV: 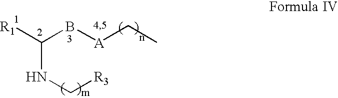
wherein
A is —CH₂CH₂—, trans —CH═CH—, or —C≡C—;
B is —CH₂—, —CH(OH)—, —CH(alkyl)—, —C(═O)—, —C(═NOH)—, or —C(═N—NH₂)—;
R₁ is CH₃, CH₂OH, CH₂SH, CH₂—NH₂, CH₂N₃, CH₂—NH—OH, CH═N—OH, CH═N—NH₂, C(═O)H, C(═O)CH₃, C(═O)CF₃, C(═O)NH₂, CH₂R₅, C(OH)R₅, or C(═O)R₅;
R₃ is H, CH₃, OH, NH₂, NH₂.HCl, NHC(═O)NH(CH₂)_{L}N(CH₃)₂, NHCONH(CH₂)_{L}N(CH₃)₂.HCl or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle (e.g., imidazolyl, tetrazolyl, pyridyl, pyrimidyl, pyrazinyl, triazolyl, morpholinyl, benzoxazyl, benzofurazyl, oxazolopyridinyl); R₆-phenyl; or R₆-condensed aromatic ring system (e.g., indenyl, naphthyl, phenanthyl, anthracenyl, fluorenyl);
R₆ is H, Cl, OH, CH₃, NO₂, NH₂, N(CH₃)₂, or NHC(═O)(CH₂)ₖCH₃;
L is an integer from 2 to10;
k is an integer from 5 to16;
n is an integer from 4 to 20;
m is an integer from 0 to 26;
and wherein the compound is not (2S, 3R, 4E), (2S, 3R, 4E), (2R, 3R, 4E), or (2R, 3R, 4E) sphingosine (R₁ is CH₂OH; B is —CH(OH)—; A is trans —CH═CH—; R₃ is H; n is 12;and m is 0);
and wherein the compound is not (2S, 3R, 4E)-N-methyl-sphingosine (R₁ is CH₂OH; B is —CH(OH)—; A is trans —CH═CH—; R₃ is CH₃; n is 12; and m is 0);
and wherein the compound is not (2S, 3R, 4E) 1-O-methyl-sphinogosine (R₁ is CH₂OH; B is —CH(OH)—; A is trans —CH═CH—; R₃ is H; n is 12; and m is 0).

In still another embodiment, the present invention provides compounds for modulation of mitochondrial ceramidase having Formula V: 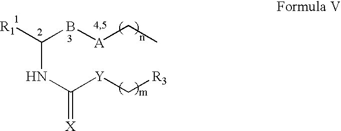
wherein
A is —CH₂CH₂—, trans —CH═CH—, or —C≡C—;
B is CH₂—, —CH(OH)—, —CH(alkyl)—, —C(═O)—, —C(═NOH)—, or —C(═N—NH₂)—;
X is O, S, or NH;
Y is NH, O, C═O, CHR₄, CH₂C═O, or CH₂CHR₄;
R₁ is a phosphate group (i.e., O—P(O)(OH)₂);
R₃is H, CH₃, OH, NH₂, NH₂.HCl, NHC(═O)NH(CH₂)_{L}N(CH₃)₂, NHCONH(CH₂)_{L}N(CH₃)₂.HCl or C(═O)R₅;
R₄is H, OH, OR₅, or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle (e.g., imidazolyl, tetrazolyl, pyridyl, pyrimidyl, pyrazinyl, triazolyl, morpholinyl, benzoxazyl, benzofurazyl, oxazolopyridinyl); R₆-phenyl; or R₆-condensed aromatic ring system (e.g., indenyl, naphthyl, phenanthyl, anthracenyl, fluorenyl);
R₆ is H, Cl, OH, CH₃, NO₂, NH₂, N(CH₃)₂, or NHC(═O)(CH₂)ₖCH₃;
L is an integer from 2 to10;
k is an integer from 5 to16;
n is an integer from 4 to 20; and
m is an integer from 0 to 26;
with the proviso that the compound is not (2S, 3R, 4E) ceramide wherein R₁ is CH₂OH; B is —CH(OH)—; X is O; Y is CHR₄; A is trans —CH═CH—; R₃ is CH₃, H, or OH; R₄ is H or OH; n is an integer from 12 to16; and m is an integer from 0 to 24.

In still yet another embodiment, the present invention provides compounds for modulation of mitochondrial ceramidase having Formula VI: 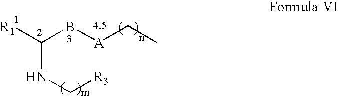
wherein
A is —CH₂CH₂—, trans —CH═CH—, or —C≡C—;
B is —CH₂—, —CH(OH)—, —CH(alkyl), —C(═O)—, —C(═NOH)—, or —C(═N—NH₂)—;
R₁ is a phosphate group (i.e., O—P(O)(OH)₂);
R₃ is H, CH₃, OH, NH₂, NH₂.HCl, NHC(═O)NH(CH₂)_{L}N(CH₃)₂, NHCONH(CH₂)_{L}N(CH₃)₂.HCl or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle (e.g., imidazolyl, tetrazolyl, pyridyl, pyrimidyl, pyrazinyl, triazolyl, morpholinyl, benzoxazyl, benzofurazyl, oxazolopyridinyl); R₆-phenyl; or R₆-condensed aromatic ring system (e.g., indenyl, naphthyl, phenanthyl, anthracenyl, fluorenyl);
R₆ is H, Cl, OH, CH₃, NO₂, NH₂, N(CH₃)₂, or NHC(═O)(CH₂)ₖCH₃;
L is an integer from 2 to10;
k is an integer from 5 to16;
n is an integer from 4 to 20; and
m is an integer from 0 to 26.

Various stereoisomers of the compounds of Formula I to VI are also encompassed by the invention except where indicated.

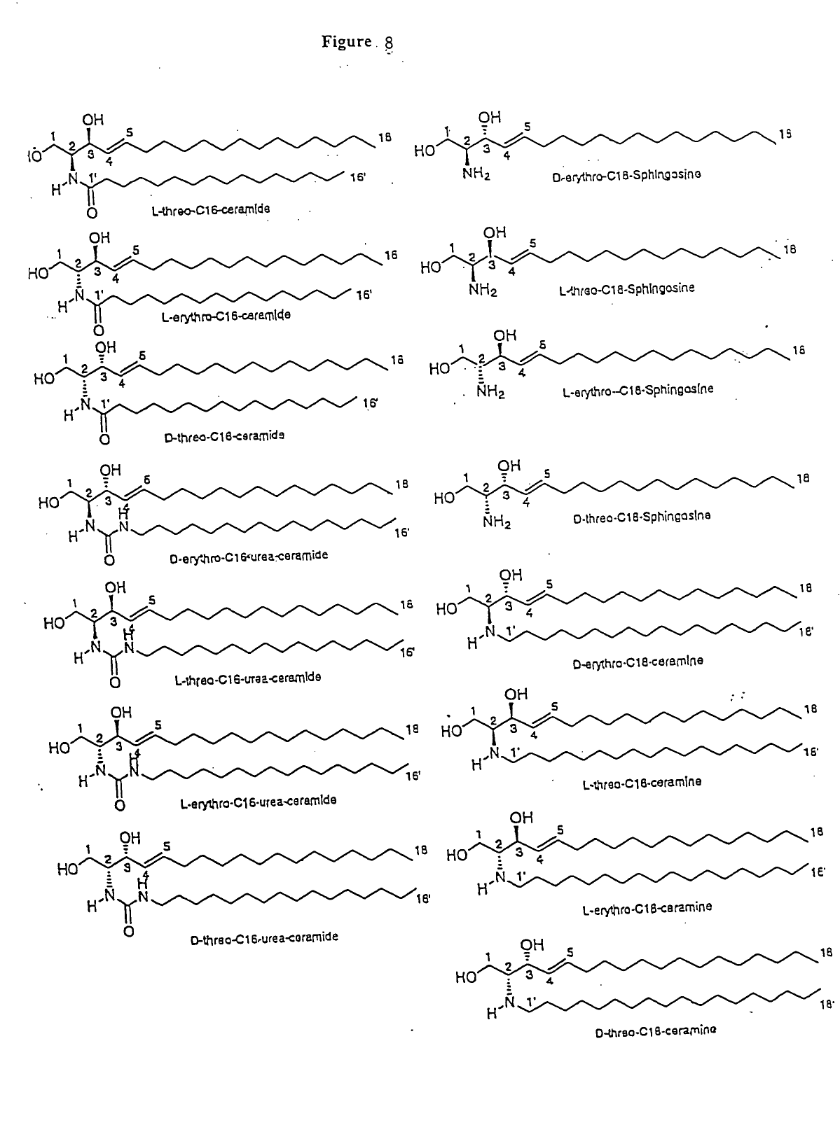

Representative compounds of the invention are shown in FIG. 8.

The preferred compounds of the present invention are inhibitors of mitochondrial ceramidase activity which include i) stereoisomers of various D-erythro-ceramides (the L-erythro-enantiomer, and the L-threo-, and the D-threo-diastereomers), ii) various 3-keto-ceramides, iii) all stereoisomers of sphingosines, iv) various N-methyl-and O-methyl-sphingosines, v) various ceramines, vi) various N,N-dimethyl-sphingosines, vii) various sphinganines and dehydrosphingosines, viii) various 3-keto-analogs of sphingosines, sphinganines and dehydrosphingosines, ix) various long chain hydrophobic primary alkyl amines, x) synthetic isosters of ceramides, C₁₆-urea-ceramides, and xi) C₆-urea-ceramide.

The more preferred compounds of the present invention are inhibitors of mitochondrial ceramidase activity which include i) ceramines, ii) N,N-dimethyl-sphingosines, iii) sphinganines and dehydrosphingosines, iv) 3-keto-analogs of sphingosine, sphinganine and dehydrosphingosine, v) long chain hydrophobic primary alkyl amines, vi) synthetic isosters of ceramide, C₁₆-urea-ceramide, and C₆-urea-ceramide.

Without being bound any theories, the inventors believe there are two theories on the different structural feature requirement for inhibition and for catalysis. First, the enzyme has two sites: 1) a catalytic site that recognizes D-erythro-ceramide in a highly stereospecific manner; and 2) a regulatory (allosteric) site that allows interaction with all stereoisomers of ceramide and sphingosine. According to this model, the un-natural isomers of ceramide (as well as sphingosine) interact at a distant site, inducing conformational changes in the enzyme that prevent interaction of the enzyme with substrate (a K_{M} type allosteric regulator). Second, catalysis of ceramide occurs by two steps. In the first step, ceramide interacts with the enzyme in a high affinity low specificity mechanism. This is supported by the ability of all stereoisomers to interact with relatively high affinity (IC₅₀ ranges between 0.11-0.26 mole %) compared to Km of hydrolysis (1.3 mole %). In the second step, catalysis occurs in a very stereochemically-specific manner such that only the D-erythro configuration allows productive interaction and catalysis.

The structural features required for initial interaction (whether at the catalytic site or at an allosteric site) are different (more general and less restrictive) than the structural features required for catalysis. If these interactions occur at the catalytic site (second possibility), then it follows that the requirements for catalysis should form a subset of the requirements for initial recognition. Thus, analogues that maintain the ability to interact (substrates and/or inhibitors) reveal modifications that are tolerated by the enzyme for initial interaction. On the other hand, analogues that function as neither substrates nor inhibitors do not interact with the enzyme, and features that are critical for molecular recognition of these sphingolipids can be found in mitochondrial ceramidase. Thus, while not necessary, in the most preferred embodiment for inhibition, the enzyme has the primary (R₁ in Formula I to IV) and secondary hydroxyl groups (B in Formula I to IV), the C4-C5 double bond (A in Formula I to IV), the trans configuration of this double bond, and either the amide or free amine.

### 5.2 Screening for Ceramidase Inhibitors

The present invention provides methods of designing and methods of screening for compounds that inhibit mitochondrial ceramidase. Based on the observations on the structure and function of inhibiting compounds, additional new compounds can be designed by making the appropriate substitutions and modifications. The inventors discovered that there were several modifications that generate potent inhibitors of the enzyme, which include: 1) amide bond modification into the urea analogue (IC₅₀: 0.33 mole %) and 2) chiral modifications at the C2 and C3 positions. Any methods known in the art for modifying the basic structure of the compounds or the invention can be used, such as but not limited to those described in Section 6. 1. Once these modifications are made by methods known in the art, a screening assay may be used to identify additional inhibitors of the mitochondrial ceramidase.

The principle of the assays involves preparing a reaction mixture of the test substance and ceramide under conditions and for a time sufficient to allow the substance to convert the ceramide into sphingosine, if the substance has any ceramidase activity. The level of ceramide or sphingosine may be detected in the reaction mixture to determine the amount of ceramidase activity present in the test substance. Many means are known in the art for assaying ceramidase activity and are within the scope of the present invention. See, for example, El-Bawab, S., Bielawska, A., and Hannun, Y. A., (1,999) J. Biol. Chem. 274, 27948-27955.

When assaying for the ability to bind or compete with wild-type ceramidase for binding to the substrate ceramide, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labelled.

### 5.3 Therapeutic use of Ceramidase Inhibitors and its Analogs

The present invention provides the uses of the compounds of the invention for treatment, prophylaxis, management or amelioration of one or more symptoms associated with various diseases and disorders. Such therapeutic compounds are ceramidase inhibitors, such as but not limited to the compounds described in the previous section under Formula I, II, III, IV, V, and VI, in Table 1 and Table 2 below, and analogs and derivatives thereof.

TABLE 1

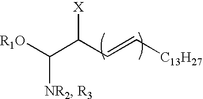

| | | | Estimated |
|---|---|---|---|
| | | Substituents: | IC₅₀ |
| Compound | Stereochemistry | X, R₁, R₂, R₃ | (mole %) |
| D-erythro-C₁₈-ceramide | (2S, 3R, 4E) | X═OH; R₁, R₂═H; | substrate |
| D-e-C₁₈-Cer | | R₃═CO(CH₂)₁₆CH₃ | |
| L-erythro-C₁₈-ceramide | (2R, 3S, 4E) | X═OH; R₁, R₂═H; | 0.26 |
| L-e-C₁₈-Cer | | R₃═CO(CH₂)₁₆CH₃ | |
| L-threo-C₁₈-ceramide | (2S, 3S, 4E) | X═OH; R₁, R₂═H; | 0.11 |
| L-t-C₁₈-Cer | | R₃═CO(CH₃)₁₆CH₃ | |
| D-threo-C₁₈-ceramide | (2R, 3R, 4E) | X═OH; R₁, R₂═H; | 0.21 |
| D-t-C₁₈-Cer | | R₃═CO(CH₃)₁₆CH₃ | |
| cis-D-erythro-C₁₆-ceramide | (2S, 3R, 4Z) | X═OH; R₁, R₂═H; | no effect |
| cis-D-e-C₁₆-Cer | | R₃═CO(CH₂)₁₆CH₃ | |
| 1-O-methyl-D-erythro-C₁₆- | (2S, 3R, 4E) | X═OH; R₁═CH₃, R₂═H; | no effect |
| ceramide | | R₃═CO(CH₃)₁₄CH₃ | |
| 1-O-Me-C₁₆-Cer | | | |
| 3-O-methyl-D-erythro-C₁₆- | (2S, 3R, 4E) | X═OCH₃; R₁, R₂═H; | no effect |
| ceramide | | R₃═CO(CH₂)₁₄CH₃ | |
| 3-O-Me-C₁₆-Cer | | | |
| 3-keto-C₁₆-ceramide | (2S, 4E) | X═O; R₁, R₂═H; | 0.6 |
| 3-keto-C₁₆-Cer | | R₃═CO(CH₂)₁₄CH₃ | |
| D-erythro-C₁₆-ceramide-1- | (2S, 3R, 4E) | X═OH; R₁═P(O)(OH)₂; | activator |
| phosphate | | R₂═H; R₃═CO(CH₂)₁₄CH₃ | |
| Cer-1-P | | | |
| D-erythro-C₁₆-urea- | (2S, 3R, 4E) | X═OH; R₁, R₂═H; | 0.33 |
| ceramide | | R₃═CONH(CH₃)₁₄CH₃ | |
| C₁₆-urea-Cer | | | |
| N-methyl-D-erythro-C₁₆- | (2S, 3R, 4E) | X═OH; R₁═H; R₃═CH₃; | no effect |
| ceramide | | R₃═CO(CH₃)₁₄CH₃ | |
| N-Me-C₁₆-Cer | | | |

TABLE 2

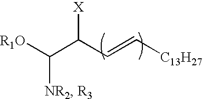

| | | | Estimated |
|---|---|---|---|
| | | Substituents: | IC₅₀ |
| Compound | Stereochemistry | X, R₁, R₂, R₃ | (mole %) |
| D-erythro-sphingosine | (2S, 3R, 4E) | X═OH; R₁, R₂, R₃═H | 0.04 |
| D-e-Sph | | | |
| L-erythro-sphingosine | (2R, 3S, 4E) | X═OH; R₁, R₂, R₃═H | 0.09 |
| L-e-Sph | | | |
| L-threo-sphingosine | (2S, 3S, 4E) | X═OH; R₁, R₂, R₃═H | 0.14 |
| L-t-Sph | | | |
| D-threo-sphingosine | (2R, 3R, 4E) | X═OH; R₁, R₂, R₃═H | 0.11 |
| D-t-Sph | | | |
| cis-D-erythro- | (2S, 3R, 4Z) | X═OH; R₁, R₂, R₃═H | weak |
| sphingosine | | | inhibitor |
| cis-D-e-Sph | | | |
| D-erythro- | (2S, 3R) | X═OH; R₁, R₂, R₃═H | 0.34 |
| dihydrosphingosine | | | |
| D-e-dh-Sph | | | |
| D-erythro- | (2S, 3R) | X═OH; R₁, R₂, R₃═H | 0.25 |
| dehydrosphingosine | | | |
| D-e-deh-Sph | | | |
| 1-O-methyl-D-erythro- | (2S, 3R, 4E) | X═OH; R₁═CH₃, R₂, R₃= | weak |
| sphingosine | | H | inhibitor |
| 1-O-Me-Sph | | | |
| 3-O-methyl-D-erythro- | (2S, 3R, 4E) | X═OCH₃; R₁, R₂, R₃═H | no effect |
| sphingosine | | | |
| 3-O-Me-Sph | | | |
| 3-keto-sphingosine | (2S, 4E) | X═O; R₁, R₂, R₃═H | weak |
| 3-keto-Sph- | | | inhibitor |
| 3-keto- | (2S) | X = O; R₁, R₂, R₃═H | 0.2 |
| dihydrosphingosine | | | |
| 3-keto-dh-Sph | | | |
| 3-keto- | (2S) | X═OH; R₁, R₂, R₃═H | weak |
| dehydrosphingosine | | | inhibitor |
| 3-keto-deh-Sph | | | |
| D-erythro-sphingosine 1- | (2S, 3R, 4E) | X═OH; R_{2,}R₃═H | activator |
| phosphate | | R₁═P(O)(OH)₂ | |
| Spb 1-P | | | |
| N-methyl-D-erythro- | (2S, 3R, 4E) | X═OH | 0.13 |
| sphingosine | | R₁, R₂═H; R₃═CH₃ | |
| N-Me-Sph | | | |
| N-stearyl-D-erythro- | (2S, 3R, 4E) | X═OH; R₁, R₂═H | 0.5 |
| sphingosine | | R₃═(CH₂)₁₇CH₃ | |
| C₁₈-Ceramine | | | |
| N,N-dimethyl-D-erythro- | (2S, 3R, 4E) | X═OH; R₁═H; R₂, R₃= | weak |
| sphingosine | | CH₃ | inhibitor |
| N,N-diMe-Sph | | | |

Ceramide modulates a number of biochemical and cellular responses to stress, including apoptosis, cell-cycle arrest and cell senescence. (For review, see Hannun et al., 2000, Trends in Cell Biol. 10:73-80; Mathias et al., 1998, Biochem. J. 335: 465-480). Several extracellular agents and stress stimuli, such as tumor necrosis factor α, chemotherapeutic agents and heat are known to cause ceramide accumulation. One approach to cause accumulation of ceramide is accomplished by regulating the activities of enzymes such as ceramidase which is involved in the metabolism of ceramide. The changes in the ceramide concentration are sufficient to reproduce many of the biological effects of cytokines and stress inducers that are coupled to ceramide accumulation. The accumulation of ceramides also reproduce many of the features of cell senescence. In many cell types, ceramides cause cell differentiation, both morphologically and through the activation of biochemical programs of cell differentiation. Ceramide also causes apoptosis in most cancer cells which can be accompanied by cell-cycle arrest. Furthermore, there is evidence which suggests that ceramide is closely associated with TNFα-induced apoptosis. Thus, according to the present invention, modulation of the levels of ceramide or sphingosine through the methods of the present invention can bring about treatment and prevention of diseases that are related to stress response and apoptosis. Several exemplary diseases and disorders are disclosed below which may be treated or prevented by the methods of the present invention.

Thus, in one embodiment, the present invention provides a method of increasing the level of ceramide in a cell comprising contacting the cell with a compound that inhibits the ceramidase activity.

In another embodiment, the invention provides a method of inhibiting the formation of sphingosine in a cell comprising contacting the cell with a compound that inhibits the ceramidase activity such that the amount of sphingosine formed as a result of conversion from ceramide is reduced.

In yet another embodiment, the invention provides a method of increasing the intracellular levels of ceramide in an animal comprising administering to the animal an effective amount of a compound that inhibits the ceramidase activity of the ceramidase protein in the animal's cells.

In yet another embodiment, the invention provides a method of inhibiting the intracellular formation of sphingosine in an animal comprising administering to said animal an effective amount of compound that inhibits the ceramidase activity of the ceramidase protein in the animal's cells.

In specific embodiments, the compound that inhibits ceramidase function are administered therapeutically or prophylactically: (1) in diseases or disorders involving an increased (relative to normal or desired) level of ceramidase protein or function, for example, in patients where ceramidase protein is biologically overactive or overexpressed; or (2) in diseases or disorders wherein in vitro (or in vivo) assays indicate the utility of ceramidase inhibitor administration. The increased level in ceramidase protein or function can be readily detected, e.g., by obtaining a patient tissue sample (e.g., from biopsy tissue) and assaying it in vitro for RNA or protein levels, structure and/or activity of the expressed ceramidase RNA or protein. Many methods standard in the art can be thus employed, including but not limited to ceramidase enzyme assays, immunoassays to detect and/or visualize ceramidase protein (e.g., Western blot, inamunoprecipitation followed by sodium dodecyl sulfate polyacrylamide gel electrophoresis, immunocytochemistry, etc.) and/or hybridization assays to detect ceramidase expression by detecting and/or visualizing ceramidase mRNA (e.g., Northern assays, dot blots, in situ hybridization, etc.), etc.

According to the invention, disorders involving cell hyperproliferation or dysfunctional sphingolipid signal transduction are treated or prevented by administration of a compound to a subject that inhibits ceramidase function. These diseases and disorders include, but are not limited to, diseases or disorders related to cell proliferation, cell attachment, cell immigration, granulation tissue development, primary and metastatic neoplastic diseases, inflammation, cardiovascular disease, stroke, ischemia or atherosclerosis. Diseases and disorders involving cell overproliferation that can be treated or prevented include but are not limited to cancers, premalignant conditions (e.g., hyperplasia, metaplasia, dysplasia), benign tumors, hyperproliferative disorders, and benign dysproliferative disorders. Cancer is characterized primarily by an increase in the number of abnormal cells derived from a given normal tissue, invasion of adjacent tissues by these abnormal cells, and lymphatic or blood-borne. Malignancies and related disorders that can be treated, prevented, managed, amerliorated, particularly metastatic cancer, by administration of a compound of the invention that inhibits ceramidase function as discussed below (for a review of such disorders, see Fishman et al., 1985, Medicine, 2d Ed., J.B. Lippincott Co., Philadelphia):

In another embodiment, disorders in which cell proliferation is deficient or is desired can be treated or prevented by administration of a compound of the invention to a subject that promotes ceramidase function.

The present invention encompasses methods for treating or preventing diseases and disorders wherein the treatment or prevention would be improved by administration of the ceramidase modulators, (i.e., inhibitors or activators) of the present invention.

In various embodiments, “treatment” or “treating” refers to an; amelioration of disease or disorder, or at least one discernible symptom thereof. “Treatment” or “treating” also refers to an amelioration of at least one measurable physical parameter associated with disease or disorder not necessarily discernible by the subject. “Treatment” or “treating” may also refer to inhibiting the progression of a disease or disorder either physically, e.g., stabilization of a discernible symptom, physiologically, e.g., stabilization of a physical parameter, or both. “Treatment” or “treating” also refers to delaying the onset of a disease or disorder. In certain embodiments, the methods and compositions of the present invention are useful as a preventative measure against disease or disorder. As used herein, “prevention” or “preventing” refers to a reduction of the risk of acquiring a given disease or disorder.

In certain embodiments, the invention provides methods for treating or preventing diseases or disorders comprising administration of a ceramidase inhibitor in combination with other treatments.

Cancers and related disorders that can be treated or prevented by methods and compositions of the present invention include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal garnmopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, flugating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/ or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In preferred embodiments, the methods and compositions of the invention are used for the treatment and/or prevention of breast, colon, ovarian, lung, and prostate cancers and melanoma and are provided below by example rather than by limitation.

The compounds of the invention that inhibits ceramidase activity can also be administered to treat premalignant conditions and to prevent progression to a neoplastic or malignant state. Such prophylactic or therapeutic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79.)

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of a compound that inhibits ceramidase function. Such characteristics of a transformed phenotype include morphology changes, looser substratum attachment, loss of contact inhibition, loss of anchorage dependence, protease release, increased sugar transport, decreased serum requirement, expression of fetal antigens, etc.

In a specific embodiment, leukoplakia, a benign-appearing hyperplastic or dysplastic lesion of the epithelium, or Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia) is indicative of the desirability of prophylactic intervention. The gene of the human ceramidase of the invention is localized on chromosome 10 (10q11)(i.e., LOC6392). Base on this location, ceramidase may be involved in diseases associated with this region, in addition to the disease and disorder discussed above, which include adenocarcinoma (thyroid), acute myeloid leukemia, and squamous cell cancer, especially that which is associated with the Nasopharynx region.

In other embodiments, a patient which exhibits one or more of the following predisposing factors for malignancy is treated by administration of an effective amount of the ceramidase inhibitors of the invention: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 197, Basic Pathology, 2d Ed., W.B. Saunders Co., Philadelphia, pp. 112-113) etc.)

The invention encompasses methods for treating or preventing a cancer or metastasis in a subject comprising in any order the steps of administering to the subject a ceramidase inhibitor. In certain embodiments, the compositions and methods of the invention can be used to prevent, inhibit or reduce the growth or metastasis of cancerous cells. In a specific embodiment, the administration of a ceramidase inhibitor inhibits or reduces the growth or metastasis of cancerous cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth or metastasis in absence of the administration of said ceramidase inhibitor.

The invention encompasses methods of disease treatment or prevention that provide better therapeutic profiles than current single agent therapies or even current combination therapies. Encompassed by the invention are combination therapies that have additive potency or an additive therapeutic effect while reducing or avoiding unwanted or adverse effects.

Other cancer treatment that may be used in combination of the administration of the ceramidase inhibitor of the present invention include the use of one or more molecules, or compounds for the treatment of cancer (i.e., cancer therapeutics), which molecules, comnpounds or treatments include, but are not limited to, chemoagents, immunotherapeutics, cancer vaccines, anti-angiogenic agents, cytokines, hormone therapies, gene therapies, biological therapies, and radiotherapies. While maintaining or enhancing efficacy of treatment, preferably the methods of the present invention increase patient compliance, improve therapy and/or reduce unwanted or adverse effects.

In a specific embodiment, the methods of the invention encompass the administration of one or more angiogenesis inhibitors such as but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates.

Additional examples of anti-cancer agents that can be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogennanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamnustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-arnino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfarn; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. These two agents are particularly useful when used in methods employing thalidomide and a topoisomerase inhibitor.

In another embodiment, the treatment of the present invention further includes the administration of one or more immunotherapeutic agents, such as antibodies and immunomodulators, which include, but are not limited to, HERCEPTIN®, RITUXAN®, OVAREX™, PANOREX®, BEC2, IMC-C225, VITAXIN™, CAMPATH® I/H, Smart M195, LYMPHOCIDE™, Smart I D10, and ONCOLYM™, rituximab, gemtuzumab, or trastuzumab.

In another embodiment, the treatment of the present invention further includes administering one or more anti-angiogenic agents, which include, but are not limited to, angiostatin, thalidomide, kringle 5, endostatin, other Serpins, anti-thrombin, 29 kDa N-terminal and 40 kDa C-terminal proteolytic fragments of fibronectin, 16 kDa proteolytic fragment of prolactin, 7.8 kDa proteolytic fragment of platelet factor-4, a 13-amino acid peptide corresponding to a fragment of platelet factor-4 (Maione et al., 1990, Cancer Res. 51:2077), a 14-amino acid peptide corresponding to a fragment of collagen I (Tolma et al., 1993, J. Cell Biol. 122:497), a 19 amino acid peptide corresponding to a fragment of Thrombospondin I (Tolsma et al., 1993, J. Cell Biol. 122:497), a 20-amino acid peptide corresponding to a fragment of SPARC (Sage et al., 1995, J. Cell. Biochem. 57:1329-), or any fragments, family members, or derivatives thereof, including pharmaceutically acceptable salts thereof.

In another embodiment, the treatment method further comprise the use of radiation.

In another embodiment, the treatment method further comprises the administration of one or more cytokines, which includes, but is not limited to, lymphokines, tumor necrosis factors, tumor necrosis factor-like cytokines, lymphotoxin-a, lymphotoxin-b, interferon-a, interferon-b, macrophage inflammatory proteins, granulocyte monocyte colony stimulating factor, interleukins (including, but not limited to, interleukin-1, interleukin-2, interleukin-6, interleukin- 12, interleukin-15, interleukin-18), OX40, CD27, CD30, CD40 or CD137 ligands, Fas-Fas ligand, 4-1BBL, endothelial monocyte activating protein or any fragments, family members, or derivatives thereof, including pharmaceutically acceptable salts thereof.

In yet another embodiment, the treatment method further comprises hormonal treatment. Hormonal therapeutic treatments comprise hormonal agonists, hormonal antagonists (e.g., flutamide, tamoxifen, leuprolide acetate (LUPRON™), LH-RH antagonists), inhibitors of hormone biosynthesis and processing, steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and antiandrogens (e.g., cyproterone acetate).

Other disorders of proliferation that may benefit from inhibition of ceramidase including cardiovascular diseases.

Vascular interventions, including angioplasty, stenting, atherectomy and grafting for the treatment of cardiovascular diseases are often complicated by undesirable effects. One of the adverse reactions to vascular intervention include endothelial and smooth muscle cell proliferation which can lead to hyperplasia, or more specifically, restenosis which is the re-clogging of the artery, occlusion of blood vessels, reperfusion injury, platelet aggregation, and calcification. In this model, an injurious stimulus induces expression of growth-stimulatory cytokines such as interleukin 1 and tumor necrosis factor. Libby et al., Cascade Model of Restenosis 1992, Circulation 86(6): III-47-III52. There is evidence which shows that ceramide inhibit the growth of endothelia and smooth muscle cells of the coronary artery.

Various therapies have been attempted to treat or prevent restenosis. However, there remains a great need for therapies directed to the prevention and treatment of cardiovascular diseases caused by hyperplasia of endothelia and smooth muscle cells. Since it has been shown that ceramide inhibit the growth of endothelia and smooth muscle cells of the coronary artery, it is therefore desirable to raise the level of ceramide for the treatment and prevention of cardiovascular diseases. Recently, Kester et al. show that ceramide used in angioplasty prevents restenosis. Kester et al., 2000, Circ. Res. 87(4):282-8. Alternative, and more effectively, one aspect of the present invention provides treatment and prevention of restenosis by adjusting the level of ceramide through administering ceramidase inhibitors.

Accordingly, it is therefore desirable to raise the level of ceramnide for the treatment and prevention of cardiovascular diseases. This can be accomplished by adjusting the intracellular level of ceramide by using the compounds and methods of the invention. The outcome of a treatment is to at least produce in a treated subject a healthful benefit, which in the case of cardiovascular diseases, includes but is not limited to a reduced risk of re-clogging of arteries after a vascular intervention procedure, and improved circulation.

In a specific embodiment, the present invention provides a method for preventing, treating, managing or ameliorating an autoimmune or inflammatory disorder or one or more symptoms thereof, said method comprising administering to a subject in need thereof a prophylactically or therapeutically effective amount of ceramidase inhibitors and a prophylactically or therapeutically effective amount of one or more immunomodulatory agents.

Interleukin-1 is a major inducer of inflammation and TNF is an important regulator of the reaction. Both cytokines can activate ceramidase, and thus inhibiting the activity of ceramidase can result in an anti-inflammatory effect. This may involve the prevention of the formation of sphingosine and sphingosine phosphate which have pro-inflammatory effects. Also, inhibition of ceramidase may prevent the hyperproliferation of immune cells that are important for inflammation. There is evidence which suggests that an increase in ceramide and a decrease in sphingosine leads to a decrease in sphingosine phosphate. Preliminary data show that in mouse fibroblast cells, L929, TNFα increases the level of ceramide and leads to PGE2 release from these cells. The release of PGE2 is also shown to be inhibited by D-(N-myristolyamino)-1-phenyl-1-propanol), D-MAPP, which is an inhibitor of one of the ceramidase. This observation may be important for inhibiting inflammatory reactions that occur in conditions, such as but not limited to rheumatoid arthritis. Thus, it is possible to treat or prevent inflammation by regulating the level of cellular ceramide using the method of the invention. As discussed above, ceramide level can be increased by administering compounds of the present invention that can inhibit mitochondrial ceramidase.

Examples of autoimmune disorders include, but are not limited to, alopecia areata, ankylosing spondylitis, antiphospholipid syndrome, autoimmune Addison's disease, autoimmune diseases of the adrenal gland, autoimmune hemolytic anemia, autoiimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (QOPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. Some autoimmune disorders are associated with an inflammatory condition. Thus, there is overlap between what is considered an autoimmune disorder and an inflammatory disorder. Therefore, some autoimmune disorders may also be characterized as inflammatory disorders.

The present invention provides methods of preventing, treating, managing or ameliorating an autoimmune or inflammatory disorder or one or more symptoms thereof, said methods comprising administering to a subject in need of a ceramidase inhibitor and one or more immunomodulatory agents. Preferably, the immunomodulatory agents are not administered to a subject with an autoimmune or inflammatory disorder whose mean absolute lymphocyte count is less than 500 cells/mm³, less than 550 cells/mm³, less than 600 cells/mm³, less than 650 cells/mm³, less than 700 cells/mm³, less than 750 cells/mm³, less than 800 cells/mm³, less than 850 cells/mm³ or less than 900 cells/mm³. Thus, in a preferred embodiment, prior to or subsequent to the administration of one or more dosages of one or more immunomodulatory agents to a subject with an autoimmune or inflammatory disorder, the absolute lymphocyte count of said subject is determined by techniques well-known to one of skill in the art, including, e.g., flow cytometry or trypan blue counts.

Examples of immunomodulatory agents include, but are not limited to, methothrexate, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators. Examples of T cell receptor modulators include, but are not limited to, anti-T cell receptor antibodies (e.g., anti-CD4 monoclonal antibodies, anti-CD3 monoclonal antibodies, anti-CD8 monoclonal antibodies, anti-CD40 ligand monoclonal antibodies, anti-CD2 monoclonal antibodies) and CTLA4-immunoglobulin. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF), anti-cytokine receptor antibodies (e.g., anti-IL-2 receptor antibodies, anti-IL-4 receptor antibodies, anti-IL-6 receptor antibodies, anti-IL-10 receptor antibodies, and anti-IL-12 receptor antibodies), anti-cytokine antibodies (e.g., anti-IFN receptor antibodies, anti-TNF-α antibodies, anti-IL-1β antibodies, anti-IL-6 antibodies, and anti-IL-12 antibodies).

Anti-inflammatory agents have exhibited success in treatment of inflammatory and autoimmune disorders and are now a common and a standard treatment for such disorders. Any anti-inflammatory agent well-known to one of skill in the art can be used in the compositions and methods of the invention. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (WNDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

The present invention also relates to the treatment of disorders involving deficient cell proliferation (growth) or in which cell proliferation is otherwise desired (e.g., degenerative disorders, growth deficiencies, lesions, physical trauma) by administering compounds that agonize, (promote) ceramidase function (e.g.,ceramide-1-phosphate and sphingosine-1-phosphate). Other disorders that may benefit from activation of cermnidase are neurodegenerative disorders (e.g., Alzheimer's disease), and disorders of aging such as immune dysfunction.

As discussed above, like treatment of neoplastic conditions, successful treatment of cardiovascular diseases, inflammation or the above-mentioned diseases can be brought about by techniques which serve to decrease ceramidase activity. Activity can be decreased by, for example, directly decreasing ceramidase gene product activity and/or by decreasing the level of ceramidase gene expression.

Techniques for the determination of effective doses and administration of such compounds are described in Section 5.4. Any technique which serves to selectively administer chemicals to a cell population of interest can be used, for example, by using a delivery complex. Such a delivery complex can comprise an appropriate chemical and a targeting means. Such targeting means can comprise, for example, sterols, lipids, viruses or target cell specific binding agents.

### 5.4 Pharmaceutical Preparation and Methods of Administration

The compounds described herein can be administered to a patient at therapeutically effective doses to treat or prevent diseases and disorder discussed above. A therapeutically effective dose refers to that amount of a compound sufficient to result in a healthful benefit in the treated subject. See, the Physician 's Desk Reference® (53^{rd} ed., 1999).

The subject to which a compound of the invention is administered is preferably an animal, including but not limited to mammal such as non-primate (e.g., cows, pigs, horses, chickens, cats, dogs, rats, etc.), and a primate (e.g. monkey such as acynomolgous monkey and a human. In a preferred embodiment, the subject is a human. The,compound of the invention can be utilized for the prevention of a variety of cancers, e.g., in individuals who are predisposed as a result of familial history or in individuals with an enhanced risk to cancer due to environmental factors.

The methods and compositions of the invention may be used in patients who are treatment naive, in patients who have previously received or are currently receiving treatment with other pharmaceutical agents or combinations, including but not limited to anti-cancer agents. Other subjects may include patients that have metastasis or no metastasis.

The methods and compositions of the invention are useful not only in untreated patients but are also useful in the treatment of patients partially or completely un-responsive to other treatments. In various embodiments, the invention provides methods and compositions useful for the treatment of diseases or disorders in patients that have been shown to be or may be refractory or non-responsive to therapies comprising the administration of other agents.

The compound of the invention can also be administered to an animal, preferably a mammal, such as farm animals and pets, to treat, prevent or ameliorate one or more symptoms associated with the disease, disorder, or infection as discussed in Section 5.3.

The absence of decreased level in ceramide protein or function can be readily detected, e.g., by obtaining a patient tissue sample (e.g., from biopsy tissue) and assaying it in vitro for ceramide. 5.4.1 Effective Dose

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. For example, the dosage can range from 10 mM to 100 μM, and preferably 1 to 10 μM. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Suitable daily doses for the treatment or prevention of a disorder described herein can be readily determined by those skilled in the art. A recommended dose of a compound of the invention is from about 0.1 mg to about 100 mg per day, given as a single once-a-day dose in the morning or as divided doses throughout the day. Preferably, a daily dose is from about 2 mg to about 25 mg per day, more preferably from about 5 mg to about 10 mg per day.

The anti-cancer activity of the therapies used in accordance with the present invention also can be determined by using various experimental animal models of such as cancer animal models such as scid mouse model or nude mice with human tumor grafts known in the art and described in Yamanaka, 2001, Microbiol Immunol 2001;45(7):507-14.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed. A lower level of proliferation or survival of the contacted cells indicates that the Therapeutic is effective to treat the condition in the patient. Alternatively, instead of culturing cells from a patient, Protocols may be screened using cells of a tumor or malignant cell line. -Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, etc.

Compounds for use in therapy can be tested in suitable animal model systems prior to testing in humans, including but not limited to in rats, mice, chicken, cows, monkeys, rabbits, etc. The principle animal models for cancer known in the art and widely used include mice:, all described in Hann et al., 2001, Curr Opin Cell Biol 2001, 13(6):778-84, which is incorporated herein by reference in its entirety.

Further, any assays known to those skilled in the art can be used to evaluate the prophylactic and/or therapeutic utility of the combinatorial therapies disclosed herein for treatment, prophylaxis, management or amelioration of one or more symptoms associated with the disease, disorder as described in Section 5.3.

Efficacy in treating inflammatory disorders may be demonstrated by detecting the ability of the ceramidase inhibitors of the present invention, or a composition of the invention to reduce or inhibit the inflammation in an animal or to ameliorate or alleviate one or more symptoms associated with an inflammatory disorder. The treatment is considered therapeutic if there is, for example, a reduction is in inflammation or amelioration of one or more symptoms following administration of the ceramidase inhibitors, or a composition of the invention.

### 5.4.2 Formulations and Use

Various delivery systems are known and can be used to administer a ceramidase modulators (i.e., inhibitors and activators) of the invention, e.g., encapsulation in liposomes, microparticles, microcapsules, receptor-mediated endocytosis (see, e.g., Wu and Wu, 1987, J. Biol. Chem. 262:4429-4432), etc. Methods of introduction include but are not limited to intradermal, intramuscular, intraperitoneal, intravenous, subcutaneous, intranasal, epidural, inhalation, insufflation (either through the mouth or the nose), oral, buccal, or rectal routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, by means of a catheter, by means of a suppository, or by means of an implant, said implant being of a porous, non-porous, or gelatinous material, including membranes, such as sialastic membranes, or fibers.

In another embodiment, the ceramidase modulator can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the ceramidase modulators can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71 :105 (1989)). In yet another embodiment, a controlled release system can be placed in proximity of the therapeutic target, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)). Other method of delivery of the therapeutics of the present invention may be used for example, as described in U.S. Pat. No. 5,679,350, which is incorporated by reference in its entirety.

The present invention also provides pharmaceutical compositions. Such compositions comprise a therapeutically effective amount of a ceramidase modulators and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the ceramidase inhibitors preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The ceramidase modulators of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the ceramidase modulators of the invention which will be effective in the treatment of a particular disorder or condition will depend on the nature of the disorder or condition, and can be determined by standard clinical techniques. In addition, in vitro assays and animal models may optionally be employed to help identify optimal dosage ranges. The precise dose to be employed in the formulation will also depend on the route of administration, and the seriousness of the disease or disorder, and should be decided according to the judgment of the practitioner and each patient's circumstances.

In specific embodiments, the ceramidase modulators of the invention are administered intramuscularly. Suitable dosage ranges for the intramuscular administration are generally about 10 μg to 1 mg per dose, preferably about 10 μg to 100 μg per dose. In one embodiment, the Therapeutic is administered in two doses, where the second dose is administered 24 hours after the first dose; in another embodiment, the Therapeutic is administered in three doses, with one dose being administered on days 1, 4 and 7 of a 7 day regimen.

Suppositories generally contain active ingredient in the range of 0.5% to 10% by weight; oral formulations preferably contain 10% to 95% active ingredient.

The invention also provides a pack or kit for therapeutic use comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or diagnostic products, which notice reflects approval by the agency of manufacture, use or sale for human administration.

Pharmaceutical compositions for use in accordance with the present invention can be formulated in conventional manner using one or more physiologically acceptable carriers or excipients.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the-compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### 6. EXAMPLE

As described in the previous sectons, ceramidases are enzymes that hydrolyze ceramides at the amide bond linking the sphingosine moiety to the fatty acids. Thus, ceramidases provide a target site for regulating ceramide-sphingosine inter-conversion. The following examples demonstrates the design, synthesis and testing of compounds that inhibit mitochondrial ceramidase.

To test the importance of the various structural features required in the enzyme-substrate interaction, a series of related compounds based on ceramide or sphingosine structures were synthesized and tested for their effect on mitochondrial ceramidase (FIG. 1). The following examples demonstrates the design, synthesis and testing of compounds that inhibit mitochondrial ceramidase which specifically hydrolyzes the D-erythro-isomer of ceramide.

### 6.1. Materials and Methods

### 6.1.1 Starting Materials and Apparatus

Stearylamine, octylamine, dodecylamine, N, N-dimethylsphingosine and general chemicals were purchased from Sigma. [9,10-³H]-Palmitic acid was purchased from American Radiolabeled Chemicals. ¹H-NMR spectra were recorded using a Bruker AVANCE 500 MHz spectrometer equipped with Oxford Narrow Bore Magnet. Chemical shifts are given in parts per million (ppm) downfield from tetramethylsilane as internal standard and the listed J values are in Hz. Mass spectral data were obtained in positive ion electrospray ionization (ESI) mode on a Finnigan LCQ ion trap mass spectrometer. Samples were infused in methanol solution with an ESI voltage of 4.5 kV and capilary temperature of 200 ° C. The purity of all synthesized lipids was >95% as estimated by TLC and ¹H-NMR analysis.

All stereoisomers of C₁₈-ceramide were prepared from their corresponding sphingosines as described previously (Bielawska, A., Crane, H. M., Liotta, D., Obeid, L. M., and Hannun, Y. A., (1993) J. Biol. Chem. 268, 26226-26232; Bielawska, A., Szulc, Z., and Hannun, Y. A., (1999) Methods Enzymol. 311, 518- 535). Sphingosines of specific stereochemical foundations [IUB nomenclature: D-erythro-(2S,3R); L-threo-; (2S,3S)] and the key intermediates: N- and 1,3-O-protected sphingosines, were utilized as basic substrates in the synthesis of the target compounds. Starting from known configurationally stable chiral auxiliary (Garner's aldehyde prepared from L-serine) we synthesized all regio- and diastereoisomeric 2S-sphingosines (Gamer, P., Park, J. M., and Malecki, E., (1988) J. Org. Chem. 53, 4395-4398; Ninkar, S., Menaldino, D., Merril, A. D., and Liotta, D., (1988) Tetrahedron Lett. 29, 3037-3040). Starting from D-serine, we had access to all remaining 2R-sphingosines (Garner, P., Park, J. M., and Malecki, E., (1988) J. Org. Chem. 53, 4395-4398; Herold, P. E., (1988) J. Org. Chem. 71, 354-362). (2S)-3-Keto-sphingoid bases (Bielawska, A., Szulc, Z., and Hannun, Y. A., (1999) Methods Enzymol. 311, 518- 535), (2S, 3R, 4E)-sphingosine-1-phosphate and (2S, 3R, 4E)-C₁₆-ceramide-1-phosphate were synthesized and characterized as described (Szulc, Z., Hannun, Y. A., and Bielawska, A., (2000) Tetrahedron Lett. 41, 7821-7824). (2S, 3R, 4E)-1-O-methyl-sphingosine was synthesized following a previously described procedure (Putz, U., and Schwarzmann, G., (1995) Eur. J. Cell Biol. 68, 113-121). The other compounds listed in Tables 1 and 2 were prepared in our laboratory as shown below.

### 6.1.1.1 Synthesis of (2S, 3R, 4Z)-sphingosine (cis-D-e-Sph)

This compound was prepared from N-Boc-4,5-dehydro-D-erythro -sphingosine in a two step synthetic sequence, as describe below, with 68% overall yield.

Part A. Catalytic reduction. A mixture of N-Boc-4,5-dehydro-D-erythro-sphingosine (0.608 g, 1.53 mmol), pyridine (0.72 mL), ethanol (7.0 mL) and Raney® 2800 nickel catalyst (0.108 g ; from Aldrich /cat. # 22,167-8/, used as a moist paste) was placed in a pressure bottle and hydrogenated on a Parr-low pressure shaking apparatus (initial pressure of hydrogen was 10.5 psi). The reaction mixture was shaken four 4 hours at room temperature. The catalyst was removed by the filtration through a Celite bead, and the filtrate was evaporated under reduced pressure to dryness. The obtained product was recrystallized from cold hexane to give pure 9:1 mixture of N-Boc-cis/trans-D-erythro-sphingosine isomers (0.586 g, 95% yield) as a white solid. TLC (Ethyl Acetate- Hexane, 2:1 ,v/v) R_{f} 0.39. 1H-NMR (400 MHz, CDCl₃) 5.79 (m, 0.1H, 5-H), 5.60 (m, 0.9H, 5-H), 5.57 (m, 0.1H, 4-H), 5.45 (m, 0.9H, 4-H), 4.63 (m, 0.9H, 3H), 4.26 (m, 0.1H, 3-H), 3.95 (m, 1H, 1-Ha), 3.76 (m,1H, 1-Hb), 3.56 (m,1H, 2-H), 2.10 (m, 2H), 1.43 (s, 9H, CH₃), 1.25 (m, 22H, CH₂), 1.0 (m, 28H, CH₂), 0.82 (t, 6H, J=7.1, CH₃).

Part B. Deprotection of the Amino-Group.

A mixture of N-Boc-cis/trans-D-erythro-sphingosine isomers (0.0935 g, 0.234 mmol), chlorotrimethylsilane (0.2 mL, 1.57 mmol) and anhydrous methanol (3.0 mL) was stirred at room temperature under a dry nitrogen atmosphere for 6 h. The solvents were evaporated under reduced pressure to dryness and the crude product was purified by flash column chromatography (elution with CHCl₃-MeOH-conc. NH₄OH, 5:1:0.05, v/v/v). The less polar fraction ws evaporated to dryness and the obtained material was recrystallized from hexane to give pure cis-D-erythro-sphingosine as a white solid (0.050 g, 71.6%; mp 75-76° C.). TLC (CHCl3-MeOH-conc. NH₄ OH, 5:1:0.05, v/v/v) R_{f} 0.45.

¹H-NMR (CDCl₃) d 5.66 (dtd, J=11.1, 8.9, 1.0, 5-H), 5.45 (ddt, J=11.2, 9.2, 1.5, 4-H), 4.43 (dd, J=8.9, 6.2, 3-H), 3.73 (dd, J=10.8, 4.5, 1-Ha), 3.68 (dd, J=10.8, 5.8, 1-Hb), 2.88 (q, J=5.6, 2-H), 2.15 (m, 2H, C(6)H₂), 1.39 (m, 2H, C(7)H₂), 1.29 (m, 20H), CH₂), 0.90 (t, J=7.1, CH₃); EI-MS (CH₃OH, relative intensity, %) m/z 599.0 (2M+H^{+,} 9), 300.1 (MH⁺, 100), 282.3 (MH^{+-H}₂O, 33), (MH⁺-2H₂O, 6). Calcd for C₁₈H₃₇NO₂ , m/z 299.3.

### 6.1.1.2 Synthesis of (2S, 3R, 4E)-N-methyl-sphingosine (N-Me-Sph)

This compound was prepared by reduction of (2S,3R)-N-Boc-4,5-dehydro-sphingosine with LiA1H₄ (Goldkoran, T., Dressler, K. A., Muindi, J., Radin, N. S., Mendelsohn, J., Menaldino, D., Liotta, D., and Kolesnik, R. N., (1991) J. Biol. Chem. 266, 16092-16097). The crude product was purified by flash column chromatography (elution with CHCl₃-MeOH-conc. NH₄OH, 4:1:0.05, v/v/v) and crystallized from cold hexane(mp 59-60° C.), TLC (CHCl₃-MeOH-conc. NH₄OH, 5:1:0.05, v/v/v; R_{f} 0.38); ¹H-NMR (CDCl₃) d 5.89 (dtd, J=15.4, 6.8, 1.0, 5-H), 5.49 (ddt, J=15.3, 5.6, 1.1, 4-H),4.75 (m, 1H, 3-H), 4.10 (dd, J=13.1, 4.6, 1-Ha), 3.97 (dd, J=13.0, 3.2, 1-Hb), 2.98 (m, 1H, 2-H), 2.85 (s, 3H, NCH₃),2.07 (q, J=7.1, C(6)H₂), 1.37 (m, 2H, C(7)H₂), 1.28 (m, 20H, CH₂), 0.88 (t, J=7.1, CH₃). El-MS (CH₃OH; relative intensity, %) m/z 649.1 ([2M+Na]⁺, 5), 325.2 ([C₂₀H₃₉NO₂]⁺, 8), 315.3 ([M+2H ]⁺, 20), 314.2 (MH⁺, 100), 296.3 ([MH^{30 —H}₂O, 28). Calcd for C₁₉H₃₉NO₂ m/z 313.3.

### 6.1.1.3 Synthesis of (2S, 3R, 4E)-3-O-methyl-sphingosine (3-O-Me-Sph)

This compound was prepared from 1-O-t-butyldiphenylsilyl-N-t-Boc-D-erythro-sphingosine (Putz, U., and Schwarzmann, G., (1995) Eur. J. Cell Biol. 68, 113-121) in a two step synthetic sequence following methods previously described for a similar class of compounds (Szulc, Z., Hannun, Y. A., and Bielawska, A., (2000) Tetrahedron Lett. 41, 7821-7824;,Kan, C. C., Ruan, Z., and Bittman, R., (1991). Biochemistry 30, 7759-7766). The crude product was purified by flash column chromatography (elution with CHCl₃-MeOH-conc. NH₄OH, 6:1:0.05, v/v/v) and isolated as a waxy semisolid (54% yield). TLC (CHCl₃-MeOH-conc. NH₄OH, 5:1:0.05, v/v; R_{f} 0.66); ¹H-NMR (CDCl₃) d 5.65 (dtd, J=15.3, 8.3, 1.2, 5-H), 5.22 (ddt, J=15.2, 8.2, 1.1, 4-H), 3.59 (dd, J=10.9, 4.7, 1-Ha), 3.52 (dd, J=10.7, 5.8, 1-Hb), 3.42 (dd, J=8.3, 6.2, 3-H), 3.18 (s, 3H, OCH₃),2.79 (q, J=5.7, 2-H), 2.0 (m, 2H, C(6)H₂), 1.42 (m, 2H, C(7)H₂), 1.19 (m, 20H, CH₂), 0.79 (t, J=7.1, CH₃); EI-MS (CH₃OH, relative intensity, %) m/z 608.5 (2M⁺—H₂O, 17), 325.2 ([C₂₀H₃₉NO₂]⁺, 17), 314.1 (MH⁺, 100), 282.2 (MH⁺—CH₃O, 65), 264.3 ([M+2H]⁺—2H₂O —CH₃, 5). Calcd for C₁₉H₃₉NO₂ m/z 313.3.

### 6.1.1.4 Synthesis of (2S, 3R, 4Z)-N-palmitoyl-sphingosine (cis-C₁₆-Cer)

This compound was prepared from (2S, 3R, 4Z)-sphingosine and palmitoyl chloride following a general acylation procedure described previously (Bielawska, A., Szulc, Z., and Hannun, Y. A., (1999) Methods Enzymol. 311, 518- 535; Jayadev, S., Liu, B., Bielawska, A. E., and Hannun, Y. A., (1995) J Biol. Chem. 270, 2047-2052. Bielawska, A., Linardic, C. M., and Hannun, Y. A., (1992) J. Biol. Chem. 267, 18493-18497). The crude was purified by flash column chromatography (elution with CHCl₃-MeOH, 94:6, v/v/) and crystallization from ethyl acetate-hexane (6:1, v/v; mp 83-85° C., 75% yield). TLC (CHCl₃-MeOH, 10:1, v/v; R_{f} 0.41); ¹H-NMR (CDCl₃), d 6.23 (d, J=7.3, NH), 5.61 (dtd, J=11.1, 8.9, 1.0, 5-H), 5.49 (ddt, J=11.2, 9.2, 1.5, 4-H), 4.63 (dd, J=8.5, 4.2, 3-H), 4.0 (dd, J=11.3, 3.6, 1-Ha), 3.84 ( m, 2-H), 3.73 (dd, J=11.4, 3.3, 1-Hb), 2.22 (t, J=7.6, COCH₂), 2.1 (m, 2H, C(6)H₂), 1.64 (m, 2H, C(7)H₂), 1.28 (m, 46H, CH₂), 0.88 (t, 6H, J=7.1, CH₃); El-MS (CH₃OH ; relative intensity, %) mvz 1097.6 ([2M-H+Na]⁺, 100), 826.5 (12), 560.6 (MNa⁺, 5), 538.2 (MH⁺, 25), 520.4 (MH⁺—H₂O, 12). Calcd for C₃₄H₆₇NO₃ m/z 537.5.

### 6.1.1.5 Synthesis of (2S, 3R, 4E)-1-O-methyl-C₁₆-ceramide (1-O-Me-C₁₆-Cer).

This compound was prepared from (2S, 3R, 4E)-1-O-methyl-sphingosine and palmitoyl chloride following the general acylation procedure described previously (Bielawska, A., Szulc, Z., and Hannun, Y. A., (1999) Methods Enzymol. 311, 518-535; Jayadev, S., Liu, B., Bielawska, A. E., and Hannun, Y. A., (1995) J Biol. Chem. 270, 2047-2052. Bielawska, A., Linardic, C. M., and Hannun, Y. A., (1992) J. Biol. Chem. 267, 18493-18497). The crude product was purified by flash column chromatography (elution with CHCl₃-MezOH, 96:4, v/v) and was isolated as a waxy semisolid (66% yield). TLC (CHCl₃-MeOH, 10:1, v/v; R_{f} 0.75); ¹H-NMR (CDCl₃): d 6.12 (d, J=6.9, NH), 5.85 (dtd, J=11.8, 7.9, 1.6, 5-H), 5.45 (ddt, J=11.2, 9.6, 1.3, 4-H), 4.13 (m, 1H, 3-H), 4.02 (m, 1H, 2-H), 3.69 (m,1H, 1-Ha), 3.55-3.46 (m,1H, 1-Hb), 3.32 (s, 3H, OCH₃), 2.21 (m, 2H, COCH₂), 2.08 (m, 2H, C(6)H₂), 1.62 (m, 2H, COCH₂C-H₂), 1.24 (m, 46H, CH₂), 0.86 (t, 6H, J=6.9, CH₃); EI-MS (CH₃OH; relative intensity, %) m/z 1125.6 ([2M-H+Na]⁺, 100), 552.3 (MH⁺, 50), 534.4 (MH⁺-H₂O, 28), 413.2 (15), 325.2 ([C₂₀H₃₉NO₂]⁺, 11), 314.2 ([M+2H]⁺—COC₁₅H₃₁, 22), 296.3 ([M+2H]⁺—H₂O—COC₁₅H₃₁, 12). Calcd for C₃₅H₆₉NO₃m.z 551.5.

### 6.1.1.6 Synthesis of (2S, 3R, 4E)-3-O-methyl-C₁₆-ceramide (3-O-Me-C₁₆-Cer)

This compound was prepared from (2S, 3R, 4E)-3-O-methyl-sphingosine and palmitoyl chloride following the same general acylation procedure (Bielawska, A., Szulc, Z., and Hannun, Y. A., (1999) Methods Enzymol. 311, 518- 535; Jayadev, S., Liu, B., Bielawska, A. E., and Hannun, Y. A., (1995) J Biol. Chem. 270, 2047-2052. Bielawska, A., Linardic, C. M., and Hannun, Y. A., (1992) J. Biol. Chem. 267, 18493-18497). The crude product was purified by flash column chromatography (elution with CHCl₃-MeOH, 96:4, v/v) and isolated as a waxy semisolid (61% yield). TLC (CHCl₃-MeOH, 10:1, v/v; R_{f} 0.70); ¹H-NMR (CDCl₃), d 6.20 (d, J=7.8, NH), 5.74 (dtd, J=11.1, 8.9, 1.0, 5-H), 5.35 (ddt, J=11.2, 9.2, 1.5, 4-H), 3.95 (m, 1-Ha), 3.90 (m, 2-H), 3.83 (dd, J=7.8, 3.7, 3-H), 3.55 (m,1-Hb), 3.25 (s, 3H, OCH₃), 2.98 (dd, J=10.2, 2.4. 1-OH), 2.21 (t, J=7.6, COCH₂), 2.06 (q, J=7.1, C(6)H₂), 1.62 (m, 2H, COCH₂CH₂), 1.38 (m, 2H, C(7)H₂), 1.21 (m, 46H, CH₂), 0.87 (t, 6H, J=7.0, CH₃). EI-MS (CH₃OH; relative intensity, %) m/z 1125.6 ([2M-H+Na]⁺, 100), 552.3 (MH⁺, 50), 534.4 (MH⁺—H₂O, 28), 314.2 (M⁺—[COC₁₅H₃₁], 22). Calcd for C₃₇H₇₃NO₃ m/z 551.5.

### 6.1.1.7 Synthesis of (2S, 4E)-3-keto-C₁₆-ceramide (3-keto-C₁₆-Cer)

This compound was prepared from D-erythro-C₁₆-ceramide by the selective oxidation of its secondary hydroxyl group following the procedure described for the N-acetyl derivative (Bielawska, A., Szulc, Z., and Hannun, Y. A., (1999) Methods Enzymol. 311, 518- 535; Jayadev, S., Liu, B., Bielawska, A. E., and Hannun, Y. A., (1995) J Biol. Chem. 270, 2047-2052; Bielawska, A., Linardic, C. M., and Hannun, Y. A., (1992) J. Biol. Chem. 267, 18493-18497). The crude product was purified by flash column chromatography (elution with CHCl₃-MeOH, 96: 4, v/v) and crystallized from acetone (mp 74-76° C., 75% yield). TLC (CHCl₃-MeOH, 10: 1, v/v; R_{f} 0.73); ¹H-NMR (CDCl₃). d=7.09 (dt, J=15.8, 7.2, 5-H), 6.71 (d, J=6.1, NH), 6.25 (d, J=15.8, 4-H), 4.88 (m, 1H, 2-H), 3.94 (m, 1H, 1-Ha), 3.79 (m, 1H, 1-Hb), 3.33 (m,1H, OH), 2.25 (m, 4H, C(6)CH₂, COCH₂), 1.63 (m, 2H, CH₂), 1.46 (m, 2H, CH₂), 1.24 (m, 44H, CH₂), 0.87 (t, 6H, J=7.0, CH₃); El-MS (CH₃OH; relative intensity, %) m/z: 1094.5 ([2M+Na]⁺, 60), 1093.4 ([2M —H+Na]⁺, 100), 948.2 (22), 558.5 (MNa⁺, 8), 535.9 (M⁺, 5). Calcd. for C₃₄H₆₅NO₃m/z: 535.5.

### 6.1.1.8 Synthesis of (2S, 3R, 4E)-N-methyl-C₁₈-ceramide (N-Me-C₁₆-Cer)

This compound was prepared from (2S, 3R, 4E)-N-methyl-sphingosine and stearoyl chloride following a general acylation procedure described previously (Bielawska, A., Szulc, Z., and Hannun, Y. A., (1999) Methods Enzymol. 311, 518- 535; Jayadev, S., Liu, B., Bielawska, A. E., and Hannun, Y. A., (1995) J Biol. Chem. 270, 2047-2052; Bielawska, A., Linardic, C. M., and Hannun, Y. A., (1992) J. Biol. Chem. 267, 18493-18497). The crude product was purified by flash column chromatography (elution with CHCl₃-MeOH, 96: 4, v/v) and crystallized from ethyl acetate ( mp. 79-81° C., 55% yield). TLC (CHCl₃-MeOH, 10: 1, v/v; R_{f} 0.42); ¹H-NMR (CDCl₃); mixture of two conformers, d =5.63 (m, 1H, 5-H), 5.40 (m, 1H, 4-H), 4.43 (m, 0.45H, 2-H), 4.12 (t, 0.55s H, J=10.2, ,3-H), 4.75 (t, 0.45H, J=10.2, 3-H), 3.97 (dd,0.45H, J=,3.6, 10.8, 1-Ha′), 3.91 (dd, 0.55H, J=3.0, 8.2, 1-Ha″), 3.83 (m, 1H, 1-Hb″, 2-H), 3.75 (dd, 0.55H, J=8.9, 10.7, 1-Hb′), 2.93 (s, 1.65H, NCH₃), 2.77 ( s, 1.35H, NCH₃), 2.44 (m, 0.9H, COCH₂), 2.33 (m, 1.1H, COCH₂), 2.03 (m, 2H, C(6)H₂), 1.61 (m, 2H, COCH₂CH₂), 1.27 (m, 50H, CH₂), 0.86 (t, 6H, J=7.0, CH₃); EI-MS (CH₃OH; relative intensity, %): m/z 1160.4 ([2M+H]⁺, 15), 1159.3 (2M⁺, 20), 893.2 ([2M+H]⁺—COC₁₇H₃₅, 22), 580.3 (MH⁺, 100), 562.3 (MH⁺—H₂O, 21), 325.2 ([C₂₀H₃₉NO₂]³⁰ , 14), 314.1([MH⁺—COC₁₇H₃₅, 52), 264.5 ([M+2H]³⁰−2H₂O—CH₃COC₁₇H₃₅, 5). Calcd for C₃₇H₇₃NO₃ m/z 579.5.

### 6.1.1.9 Synthesis of (2S, 3R, 4E)-N-stearyl-sphingosine (C₁₈-ceramine)

This compound was synthesized by the reduction of the amido group of (2S, 3R, 4E)-C₁₈-ceramide following the procedure described previously (Goldstein, A. S., Lukyanov, N. A., Carlson, P. A., Yager, P., and Gelb, M. H., (1997) Chem. Phys. Lipid 88, 21-36). The crude product was purified by flash column chromatography (elution with CHCl₃-MeOH-conc. NH₄OH, 8:1:0.05, v/v/v) and isolated as a waxy semisolid (54% yield). TLC (CHCl₃-MeOH-conc. NH₄OH, 5:1:0.05 v/v/v; R_{f} 0.83); ¹H-NMR (CDCl₃), d 5.75 (dtd, J=15.3, 6.3, 1.2, 5-H), 5.48 (ddt, J=15.2, 6.6, 1.1, 4-H), 4.23 (m, 1H, 3-H), 3.69 (m, 2H, 1-H), 2.67 (m, 2H, NHCH₂), 2.61 (m, 1H, 2-H), 2.05 (q, 2H, J=7.1, C(6)H₂), 1.50 (m, 2H, NHCH₂CH₂), 1.25 (m, 52H, CH₂), 0.86 (t, 6H, J=6.9, CH₃). EI-MS (CH₃OH; relative intensity, %) m/z 552.6 (MH⁺, 100), 325.2 ([C₂₀H₃₉NO₂]⁺, 12). Calcd for C₃₆H₇₃NO₂ m/z 551.5.

### 6.1.1.10 Synthesis of (2S, 3R,4E)-N-[2-(1,3-Dihydroxy-4E-octadecene)]-N′-hexadecane-urea (C₁₆-urea-Cer)

To a solution of (2S, 3R, 4E)-sphingosine (43.5 mg, 0.145 mmol) in anhydrous acetonitrile (4 mL) and anhydrous diethyl ether (3 mL), hexadecyl isocyante (49.2 mg, 0.181 mmol) was added, and the mixture was stirred at room temperature under nitrogen for 4 h. After evaporation of the solvents under a reduced pressure, residue was crystallized from acetone-methanol (1:3; v/v) to give a pure urea isoster of ceramide as a white microcrystalline powder (mp. 105-106° C., 68.1 mg, 83% yield); TLC (CHCl₃-MeOH, 10:1, v/v; R_{f} 0.39); ¹H-NMR (MeOD-CDCl₃), d 5.46 (dtd, J=15.4, 6.7, 1.4, 5-H), 5.35 (ddt, J=15.4, 6.9, 1.3 4-H), 3.87 (m, 1H, 3-H), 3.41 (m, 2H, 1-Ha, 2-H), 3.37 (dd, J=10.2, 3.4, 1-Hb), 2.85 (m, 2H, NHCH₂), 1.78 (m, 2H, C(6)H₂), 1.20 (m, 2H, NHCH₂CH₂), 1.0 (m, 48H, CH₂), 0.62 (t, 6H, J=7.2, CH₃). EI-MS (CH₃OH; relative intensity, %) m/z: 1134.4 ([2M+H]⁺, 31), 1133.4 (2M⁺, 43), 567.3 (MH⁺, 100), 549.4 (MH⁺—H₂O, 67), 409.3 (18), 264.3 ([M+2H]⁺—2H₂O—CONHC₁₆H₃₃, 8). Calcd for C₃₅H₇₀N₂O₃m/z 566.6.

### 6.1.1.11 Synthesis of (2S,3R,4E)-N-[2-(1,3-dihydroxy-4E-octadecene)]-N′-hexane-urea(C₆-Urea-Cer)

To a solution of (2S,3R,4E)-sphingosine (84 mg, 0.28 mmol) in anhydrous acetonitrile (3 mL) and anhydrous chloroform (3 mL) hexyl isocyante (0.082 mL, 0.56 mmol) was added, and the mixture was stirred at room temperature under a dry nitrogen for 2 hours. After evaporation of the solvents under reduced pressure on rotary evaporator, the residue was purified by flash column chromatography (elution with CHCl₃-MeOH, 50: 4, v/v) following crystallization from acetone-ethyl acetate (1:1, v/v) to give a pure C6-urea-ceramide as a white microcrystalline powder (mp.95-97° C., 89.1 mg, 75% yield); TLC R_{f} (CHCl₃-MeOH, 10: 1, v/v): 0.28.

1H-NMR (500 MHz, MeOD-CDCl₃) 5.38 (dtd, J=15.3, 6.5,1.3, 5-H), 5.35 (ddt, J=15.3, 6.7,1.2 4-H), 3.77 (m, 1H, 3-H), 3.40 (m, 2H, 1-Ha, 2-H), 3.35 (dd, J=10.1, 3.5, 1-Hb), 2.80 (m, 2H, NHCH₂), 1.72 (m, 2H, CH₂), 1.18 (m, 2H, NHCH₂ CH₂), 1.0 (m, 28H, CH₂), 0.60 (t, 6H, J=7.1, CH₃); EI-MS (CH₃OH; relative intensity, %) m/z: 875.4 ([2M+Na]⁺, 40), 853.4 ([2M+H]⁺, 55), 554.3 (15), 427.1 (MH⁺, 92), 409.3 (100). Calcd. for C₂₅H₅₀N₂O₃ m/z 426.3.

### 6.1.1.12 Synthesis of Radiolabeled Compounds

(2S,3R,4E) [N-9,10-³H]-Palmitoyl-sphingosine: [N-³H]-C₁₆-ceramide was prepared as described (Bielawska, A., and Hannun, Y. A., (1999) Methods Enzymol. 311, 499-518). [3-³H] (2S,3R)-N-[2-(1,3-dihydroxy-4E-octadecene)], N′-hexadecanene-urea: [3-³H]-C₁₆-urea-Cer) was prepared from [3-³H]-sphingosine (29) and hexadecyl isocyanate following the procedure described for its non-radioactive analog.

### 6.1.2 Enzymatic Assays

Enzyme assays can be used to detect or measure the ceramidase activity of a test substance. The test substance may be a patient sample, cell lysate, a purified preparation of the enzyme, a mutant, a variant, or an analog of ceramidase. This is useful in evaluating whether a given substance has ceramidase activity. In one embodiment, the inhibitory compounds of the invention can be used as a positive control in such enzyme assays. The compounds can also be used to identify and distinguish various forms of ceramidases based on their relative effectiveness in inhibiting these other forms of ceramidases.

The enzymatic activity of mitochondrial ceramidase was determined by either one of the two following methods:

a. Radioactive assay: The mt-CDase activity was determined by measuring the release of radioactive fatty acid from tritiated ceramide ([³H]-C₁₆-Cer), labeled in the acyl chain. Briefly, organic solutions of ceramide and its analogues were initially mixed together, solvents were completely evaporated under nitrogen, then the dried lipids were dispersed in Triton-X 100 by sonication and vigorous vortexing. The reaction assay contained (in a final volume of 200 μl): enzyme (5-10 ng) and 10 nmoles of tritiated C₁₆-ceramide (1×10⁵ dpm) delivered in 100 μl of 1% Triton X-100 micelles in a glycine buffer pH 9.5 (200 mM). The reaction mixture was incubated at 37° C. for 1 hour ( the reaction was linear for at least 2 hours) and terminated with the consecutive addition of 2 ml of isopropyl alcohol: heptane: 1N NaOH solution (4:1: 0.1 v/v/v), water (1 ml) and heptane (1 ml). Following phase separation by centrifugation, the lower layer was washed twice with 1 ml heptane, then sulfuric acid (1 ml,1N) and heptane (2 ml) were added, and the upper phase containing the released fatty acid was counted (hydrolyzed [³H]C₁₆-ceramide was always less than 10%).

b. HPLC-assay: The Mt-CDase activity was also determined by measuring the amount of sphingosine released. Basically the assay was initially the same as described above but using non-radioactive ceramide. The reaction was stopped with 800 μl of chloroform: methanol (2:1 v/v) mixture followed by the addition of phyto-sphingosine (0.5 nmoles) and dihydro-sphingosine (1 nmole) as internal standard and carrier, respectively. The reaction mix was then extracted with NaOH (50 μl, 1N), and the lower chloroform layer containing the sphingosine was further washed with water (100 μl) and dried. The released sphingosine was quantified as the ortho-phthaldehyde (OPA) derivatives as described (30). Briefly, HPLC analysis was conducted using a Waters 501 HPLC pump model with a 5 μm C₁₈ Ultrasphere ODS Beckmann column (4.6 cm×25 cm) with a C₁₈-guard column. The solvent system was methyl alcohol: potassium phosphate buffer (5 mM), pH 7.0 (90:10 v/v) at a flow rate of 1 ml/min. A Shimadzu RF-551 spectrofluorometer detector was used with excitation and emission wavelengths of 340 nm and 455 nm, respectively. The retention times were 12, 18 and 26 min for phyto-sphingosine, sphingosine and dihydrosphingosine respectively. Activity was determined relative to the phytosphingosine OPA peak.

The effects of various compounds of the invention which are stereochemical and structural analogues of ceramide and sphingosine on mt-CDase were investigated. Hydrolysis of D-erythro-C₁₆ or D-erythro-C₁₈-ceramide at a concentration of 0.625 mole % (50 μM) by mt-CDase was determined in the presence of different concentrations [0-1.25 mole % (0-100 μM)] of compounds listed in Tables 1 and 2. Results were expressed as percent of the control ceramidase activity.

### 6.1.3 In Vitro Cell-Based Assay

MCF7 human breast carcinoma cells (ATCC No. HTB-22) and human embryonal kidney cells HEK-293 (ATCC No. CRL-1573) were purchased from American Type Culture Collection.

To investigate the effect of the compound of interest on each of these cell lines, on Day 1, about 50 μL (about 5,000 cells) of the MCF7 or HEK-293 cells were plated down into 96-well plate which contains RPMI 1640 1× (MOD) media (purchased from Media Tech) containing L-glutamine and 10% fetal calf serum (purchased from Gibco). The cells were contained at 37° C. in a humidified atmosphere containing 5% CO₂ overnight. On Day 2, the cells were treated with the compound of interest which was dissolved in 100% ethanol. To facilitate dissolution of the compound of interest, a 98% ethanol/2% dodecane can be used as the solvent instead of 100% ethanol. The control is a sample without the compound of interest. The cells were treated with the compound of interest for 18 hours after which the effect on cell viability was measured by MTT assay (Mossman, T. (1983) J. Immuno. Meth. 65, 55-63; and Berridge, M. V. and Tan, A. S. (1993) Archives of Biochemistry & Biophysics, 303, 474-482) as described below.

MTT Assay:
(1) Add to each well 25 μL of MTT stock solution (5 mg/mL in PBS);
(2) Incubate for 5 hours;
(3) Add 100 μL of lysing buffer (20% SDS (w/v), 50% N,N-Dimethyl formamide (v/v)) and stand overnight;
(4) read plate at 595 nm.

### 6.2. Results: Biochemical Activity

### 6.2.1 Modifications of Ceramide

### 6.2.1.1 Stereochemical modification at the C2-C3-chiral centers; Effects of the Optical Isomers

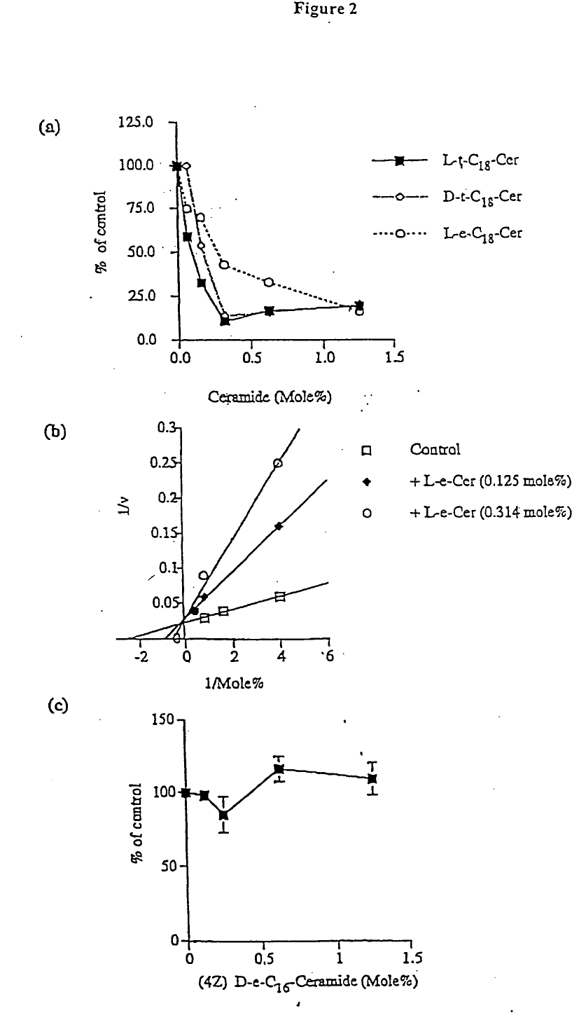

Initially the effect of ceramide stereoisomers on the hydrolysis of D-e-Cer by mt-CDase was investigated. The enzyme was assayed at a constant substrate (D-e-C₁₈-Cer) concentration, 0.625 mole % (50 μM), and in the presence of varying concentrations, 0-1.25 mole % (0-100 μM), of ceramide stereoisomers. Enzyme activity was determined by measuring the amount of sphingosine released using the HPLC assay. All ceramide stereoisomers at 1.25 mole % (100 μM) inhibited mt-CDase activity to the same extent (77%) (FIG. 2a). At lower concentrations, however, the threo isomers showed more inhibitory effects than the erythro isomer. A 50% inhibition of mt-CDase activity was obtained at 0.11, 0.21, and 0.26 mole % (8.8, 16.6, and 20 μM) for L-threo, D-threo and L-erythro, respectively (Table 1). The type of the inhibition demonstrated by the enantiomer of the natural substrate was also tested. Ceramidase was then assayed in the presence of different constant concentrations of L-erythro-C₁₈-ceramide, 0.125 and 0.314 mole % (10 μM and 25 μM), while varying D-e-C₁₈-Cer concentration from 0 to 2.5 mole % (0-200 μM). An increase in the Km value was observed with no change in Vmax (FIG. 2b) indicating a competitive type of inhibition by the L-erythro-C₁₈ enantiomer. Therefore, whereas mt-CDase only hydrolyzes the natural D-erythro isomer, all other stereoisomers were inhibitory, with the L-threo-isomer being the most potent. These findings confirm the stereospecificity of the enzyme to its substrate.

### 6.2.2 Effect of the Geometrical C4-C5 cis isomer

The effects of the cis D-erythro-C₁₆-ceramide, the 4-Z isomer, on ceramidase activity were evaluated. This compound was prepared as described in “Experimental Procedures”. Using HPLC analysis, ceramidase was assayed with (4E) D-erythro C₁₆-ceramide (0.625 mole %) in the presence of varying amounts of the 4Z isomer: 0-1.25 mole % (0-100 μM). Results presented in FIG. 2c show no significant effect of the cis isomer on substrate hydrolysis, suggesting an important role for the trans double bond in the interaction of ceramide with ceramidase.

### 6.2.2.1 Modification of the Functional Groups

Ceramide is characterized by several potentially reactive centers, which include two hydroxyl groups, a double bond in the sphingosine back bone and an amide bond. The role of these structural elements on ceramidase activity was investigated.

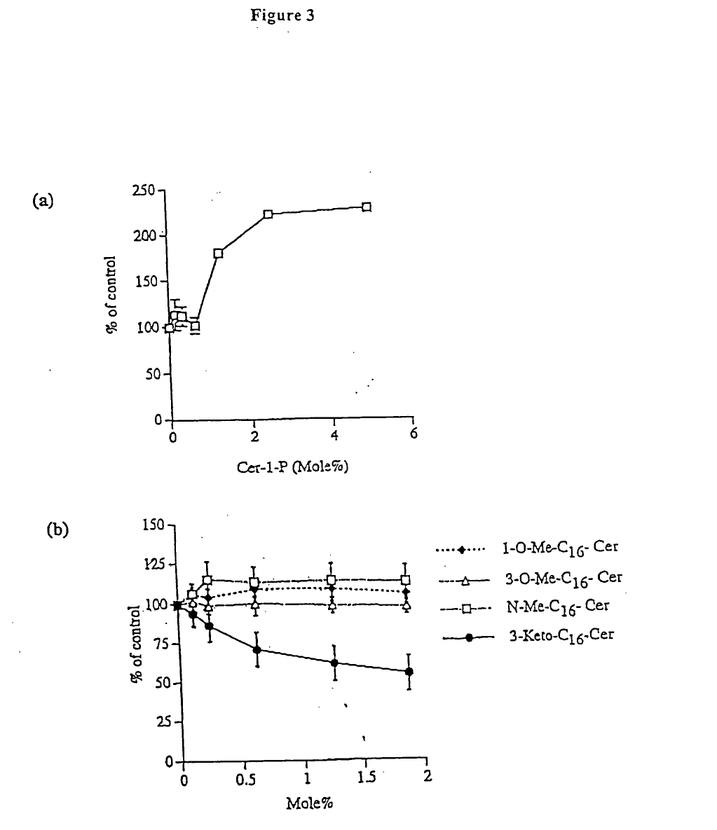

i)Phosphorylation: Ceramide has a primary (C₁) and a secondary (C₃) hydroxyl groups. The phosphorylation of the primary hydroxyl group yields ceramide-1-phosphate (Cer-1-P), which appears to exist in-vivo. The effects of varying concentrations (0-5 mole %; 0-400 uM) of Cer-1-P on the hydrolysis of [³H]-C₁₆-ceramide (0.625 mole %) were determined. A significant increase (220%) in ceramide hydrolysis was observed (FIG. 3a), indicating that phosphorylation of the primary hydroxyl group not only prevents hydrolysis of the molecule, but actually enhances ceramidase activity.

ii) O-Methylation: The effect of the O-methylated-ceramides on mt-CDase activity was studied. Both, 1-O-methyl and 3-O-methyl ceramides were synthesized as described Section 6.1. Their effect on mt-CDase activity was tested at constant D-e-C₁₆-Cer (0.625 mole %) while varying the concentration of 1- and 3-O-methyl ceramides from 0-1.88 mole % (0-150 μM). FIG. 3b shows that there is no effect of either compound on ceramidase activity, indicating that methylation of either of the hydroxyl groups inhibits the interaction of ceramide with the enzyme.

iii) Oxidation: The effect of the oxidized secondary hydroxyl group (C3) of ceramide on ceramidase activity was investigated. 3-Keto analogue of D-e-C₁₆-Cer served as a substrate, and when tested against ceramide as a substrate, it behaved as a competitive substrate with a maximal inhibition (53%) at 0.625 mole % (50 μM) of 3-ketoceramide (FIG. 3b).

iv) N-Methylation: The effect of the introduction on of N-methyl group into the secondary amide function of ceramide on mt-CDase was tested. Enzyme activity was assayed in the presence of constant [³H]-D-erythro-C₁₆-ceramide (0.625 mole %) while varying the concentration of N-methyl-C₁₆- ceramide (0-1.8 mole %). Results shown in FIG. 3b indicate that N-methyl-ceramide did not affect ceramidase activity. Thus, replacement of hydrogen in NHCO-function of ceramide with a methyl group prevented its interaction with mt-CDase. This may suggest an important role of the free amido hydrogen in ceramide hydrolysis, but also can raise the issue of steric effects imparted by the bulkier methyl group.

In conclusion our data suggest the involvement of the primary and the secondary hydroxyl groups and the free amido hydrogen of ceramide in the interaction of ceramide with ceramidase.

### 6.2.2.2 Effects of Products of Ceramidase on mt-CDase Activity

Ceranidase action on D-erythro-C₁₆-ceranide yields palmitic acid and sphingosine. Their effects on mt-CDase activity were investigated.

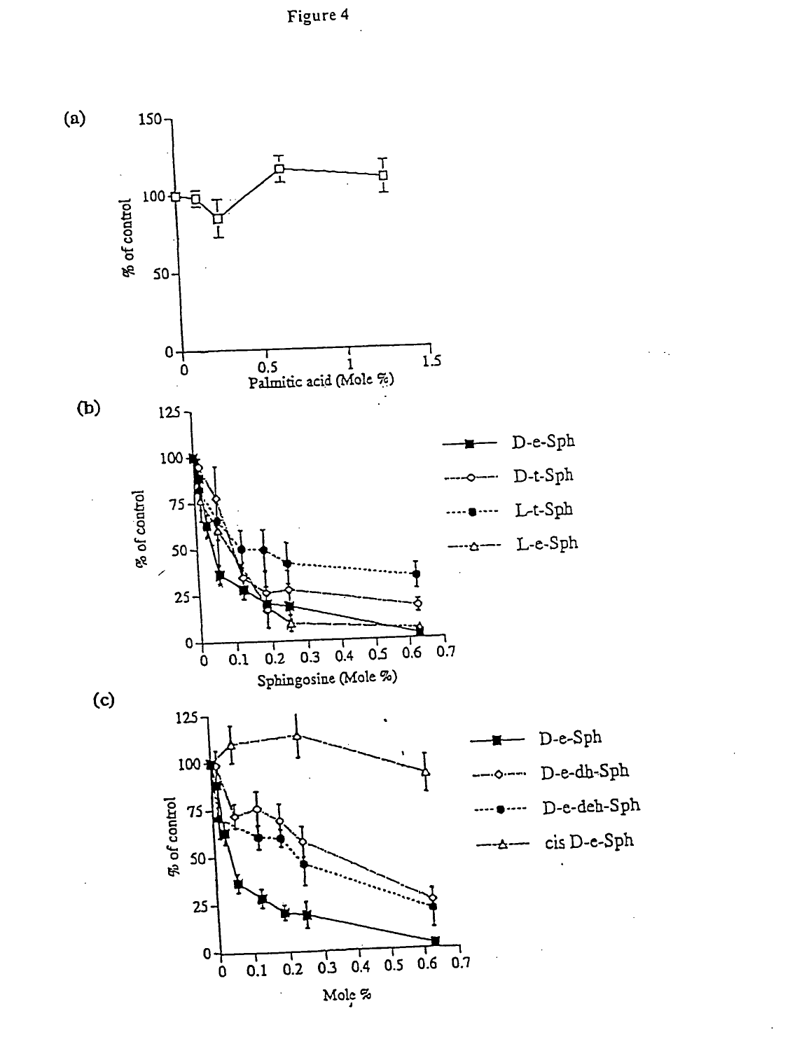

i) Effect of palmitic acid: the enzyme was assayed using [³H]-D-erythro-C₁₆-ceramide 0.625 mole % (50 μM) as a substrate and in the presence of varying palmitic acid concentrations: 0-1.25 mole % (0-100 μM). Activity was then determined by measuring the amount of [³H]-fatty acid released. FIG. 4a showed that there is no significant variation in the fatty acid released with increasing concentration of palmitate, indicating that fatty acids exert no negative feed-back effects on ceramidase.

ii) Effect of sphingosine: The effects of D-erythro-sphingosine (0-0.625 mole %), the second product of ceramide hydrolysis, on ceramidase activity were studied. Unlike palmitic acid, D-erythro-sphingosine inhibited ceramide hydrolysis in a concentration dependent manner with an IC₅₀ of 0.04 mole % (3.3 μM), and a maximal inhibition (98%) at D-erythro-sphingosine concentration of 0.625 mole % (50 μM) (FIG. 4b). To determine whether the inhibitory effect of D-erythro-sphingosine displays stereospecificity, other sphingosine stereoisomers were tested. FIG. 4b shows that whereas all stereoisomers of D-erythro-sphingosine followed a similar inhibitory pattern at low concentrations, 0.015-0.125 mole % (1-10 μM), the extent of inhibition, however, by the L-erythro (98%) isomer was more than the threo diastereomers (60% for L-threo and 75% for D-threo) at higher concentrations.

In conclusion, whereas fatty acids have no effect, all sphingosine stereoisomers inhibited ceramidase. This suggests a possible negative feed back regulatory role of sphingosine on ceramidase.

### 6.2.3 Modifications of Sphingosine

a) Effect of saturation and desaturation at the C4-C5 position. A distinctive feature of sphingosine and ceramide is the rigid 4E trans double bond that joins the carbon chain to the chiral backbone. Consequently, the role of this double bond was investigated. Towards this objective, three different variations of the C4-C5 position were synthesized and examined: the alkene-containing D-erythro-sphingosines (E, C4═C5 and Z, C4═C5); the reduced sphingosine (C4-C5); namely, D-erythro-dihydrosphingosine (D-e-dh-Sph); and the alkyne (C4═C5 ) analogue of sphingosine, D-erythro-dehydrosphingosine (D-e-deh-Sph). All analogs of sphingosine inhibited the mt-CDase activity in a concentration dependent manner, 0-0.625 mole % (0-50 μM), with D-e-Sph being the most potent and D-e-dh-Sph the least potent (FIG. 4c). The estimated IC₅₀ values were 0.04, 0.25, and 0.34 mole % for D-e-Sph, D-e-deh-Sph and D-e-dh-Sph, respectively. These results indicate that the presence of the double bond, though not necessary, increases significantly the extent of inhibition of ceramidase.

b) Effect of the cis isomer of D-erythro-sphingosine: Since the naturally occurring sphingosine is a trans isomer, the effect of the cis isomer of sphingosine on mt-CDase activity was tested. Contrary to the significant inhibition in ceramidase activity by the trans isomer, the cis isomer had no effect at concentrations lower than 0.5 mole % (FIG. 4c). FIG. 4c shows that at equimolar concentration, 0.625 mole % (50 μM), the trans isomer inhibited (98%) ceramide hydrolysis significantly whereas the cis isomer resulted only in 10% inhibition. A maximal inhibition of 32% was obtained by the cis isomer at higher concentrations, greater than 2 mole % indicating that the cis isomer is a less potent inhibitor than its natural geometric isomer and suggesting a role for the orientation of the trans double bond in interaction with the enzyme.

c) Effect of the modified hydroxyl groups:

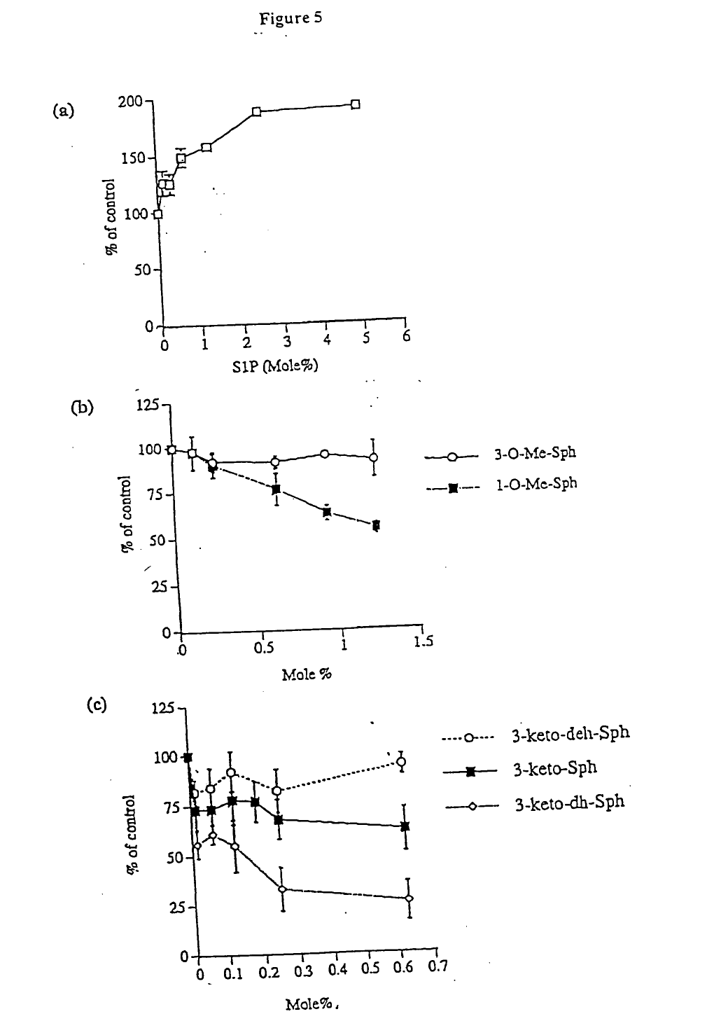

i) Phosphorylation: Sphingosine-1-phosphate (S-1-P) is a natural product of sphingolipid metabolism, thus its effect on the hydrolysis of tritiated C₁₆-ceramide (0.625 mole %) was investigated. Similar to Cer-1-P, S-1-P tested over a wide range of concentrations, 0-5 mole % (0-400 μM), increased (180%) the hydrolysis of ceramide (FIG. 5a). Therefore, contrary to the effect of sphingosine on ceramidase activity, phosphorylation of the primary hydroxyl group of sphingosine not only abolishes the inhibitory effect by sphingosine but also enhances ceramide hydrolysis.

ii) O-Methylation: Modification of the primary and the secondary hydroxyl groups of sphingosine by methylation into the corresponding 1-O-methyl and 3-O-methyl sphingosines, respectively was performed as described in Section 6.1. Both compounds were then tested for their effect on mt-CDase at 0-1.25 mole % (0-100 μM). No significant inhibition by either compound was observed (FIG. 5b). At 0.6 mole %, a concentration where D-e-sphingosine totally inhibited the activity, 3-O-Me-Sph had no effect whereas 1-O-Me-Sph inhibited only by ˜25%. In conclusion, O-methylation of sphingosine at the hydroxyl group in position 3 abolishes the inhibitory effect of sphingosine, and the O-methylation at the C1 position severely affects the inhibitory effect of sphingosine.

iii) Oxidation: Results in FIG. 3b showed that the 3-keto-ceramide inhibits ceramidase activity. To determine whether this is due to the keto-sphingoid backbone, the corresponding keto analogues of D-e-Sph, D-e-dh-Sph and D-e-deh-Sph were synthesized and their effects on ceramidase were determined. Surprisingly, compared to their non-oxidized analogues, the keto-compounds followed an opposite profile. Whereas D-e-dh-Sph was the least potent (FIG. 4c), its keto analogue had the most potent effect among the keto-compounds tested. A maximal inhibition of 75% was obtained at 0.625 mole %, (FIG. 5c). On the other hand, contrary to the significant inhibitory effect of sphingosine and dehydrosphingosine (98% and 75%; FIG. 4c) on mt-CDase, their corresponding 3-keto derivatives had relatively small effects (FIG. 5c). A maximal inhibition of 15% and 35% for 3-keto-deh-Sph and 3-keto-Sph, respectively, was obtained at 0.625 mole % (50 μM). Our results indicate that the oxidation of the secondary alcohol to the keto group reduces the inhibitory effect of sphingosine but enhances that of dihydrosphingosine.

iv) Role of the free NH-amino-function on CDase activity: effect of N-alkyl-sphingosines

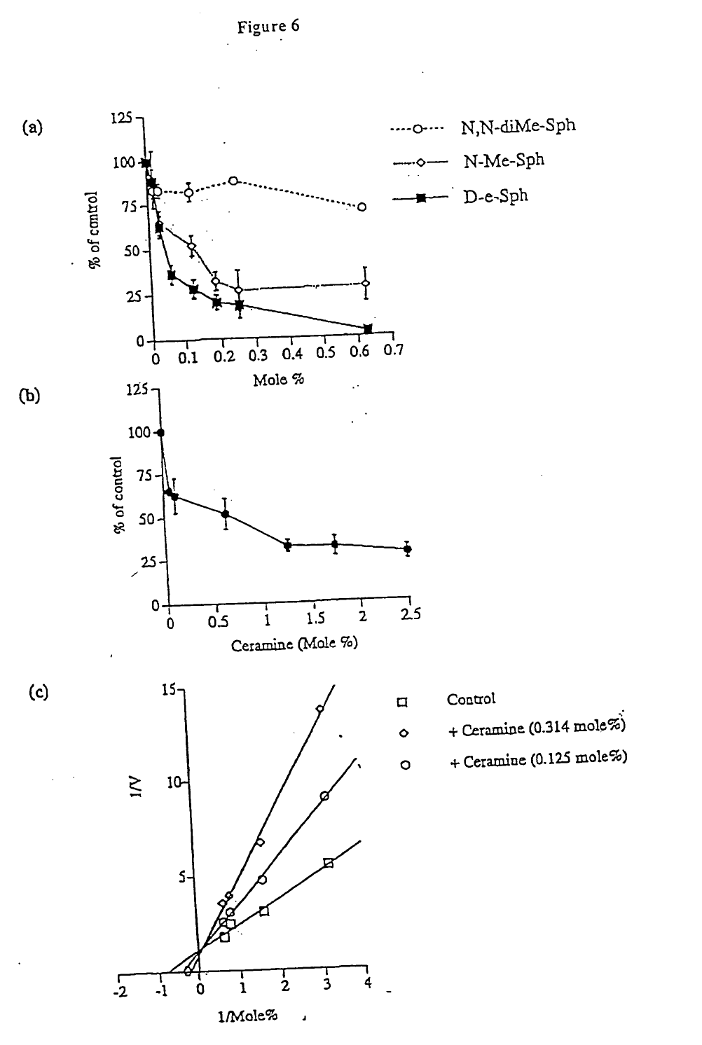

The results on mt-CDase inhibition by sphingosine raise the question of whether the inhibition may be attributed to the primary amine group of sphingosine. Consequently, the effect of N-alkylated sphingosines on enzyme activity was investigated. FIG. 6a shows that whereas N-Me-Sph exhibited an inhibitory effect (75%), further methylation reduced the extent of inhibition significantly. Even at a concentration 10-fold that of N-methyl-sphingosine (2.5 mole %), N,N-diMe-Sph resulted in a maximal inhibition of only 30%. Therefore, inhibition by sphingosine is significantly reduced by N,N-dimethylation. It may suggest an important role of the free amino hydrogen of sphingosine in the interaction with the enzyme, but also can raise the issue of steric effects of the larger methyl groups.

N-stearyl-sphingosine (C₁₈-ceramine), a long chain homolog of N-methyl-sphingosine also inhibited ceramidase activity significantly (75%) at 1.25 mole % FIG. 6b. The inhibition by ceramine (0.125 and 0.314 mole %) increased the Km but had no effect on the Vmax, indicating that it is a competitve inhibitor of ceramidase (FIG. 6c). These results confirm further the role of the free N—H group and identify ceramine as a competitive inhibitor of mt-CDase.

### 6.2.4 Effect of Urea Isoster of Ceramide on mt-CDase Activity

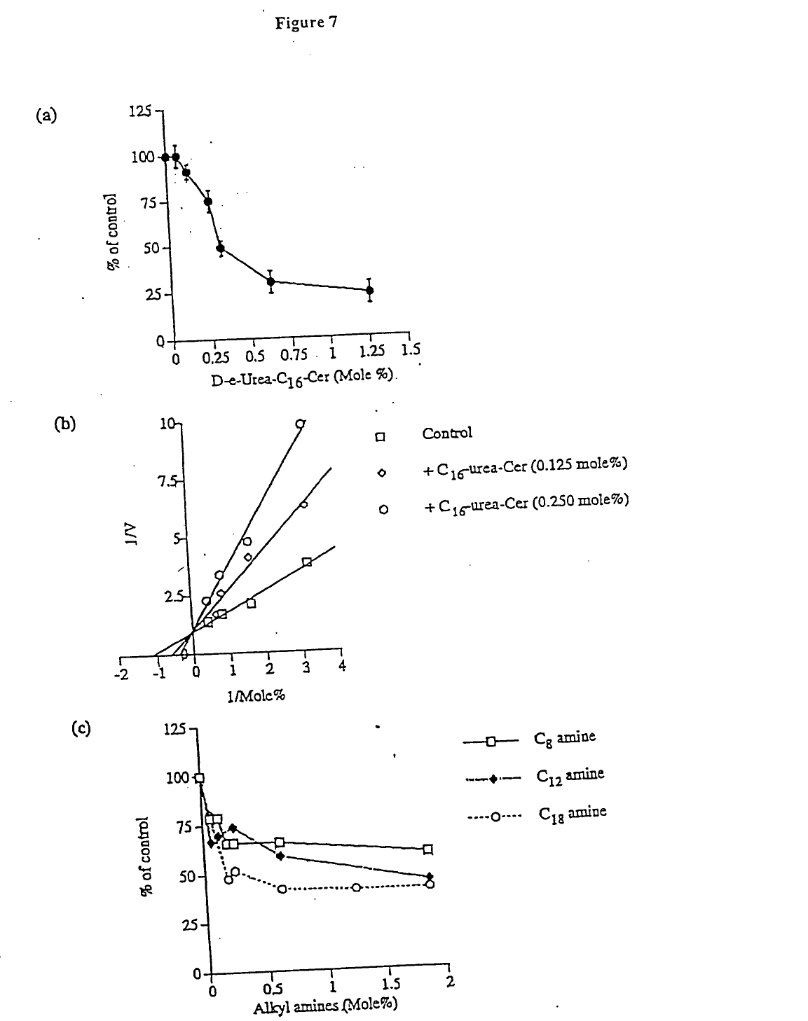

The amide bond in ceramide introduces another rigid element to the molecule linking the sphingosine backbone to the fatty acyl chain. Moreover, the possible existence of inter-/intra-molecular hydrogen bonding between the hydrogen of the amido group NHCOR and the hydroxyl groups in ceramide may influence ceramide-enzyme interactions as shown for N-methylated ceramide. The amide function of ceramide is further modified: NHCOR to its urea isoster NHCONHR, namely urea-ceramide (C₁₆-urea-Cer). Ceramidase activity was determined at constant D-e-C₁₆-cer 0.625 mole % (50 μM) while varying the concentration of C₁₆-urea-Cer 0-1.25 mole % (0-100 μM). A significant inhibition (75%) in ceramidase activity was obtained at 0.625 mole % (50 μM) with an IC₅₀ of 0.31 mole % (25 μM) (FIG. 7a). To determine the type of inhibition, ceramidase was assayed in the presence of 0.125 and 0.25 mole % (10 and 20 μM) of C₁₆-urea-Cer while varying D-e-C₁₆-Cer concentration (0.1-2.5 mole %). FIG. 7b is a kinetic analysis by double reciprocal plots. It shows an increase in the Km value with no variation in the maximal velocity, indicating that C₁₆-urea-Cer is a competitive inhibitor of ceramidase with respect to the ceramide substrate.

However, the presence of the two potentially hydrolyzable amide bonds [Sph-NH^{a}—CO^{b}—NH—R] in the urea-isoster raises the question if the observed inhibition is due to D-erythro-C₁₆-urea-ceramide acting as a competitive substrate or possibly even due to the released sphingosine or N-palmitylamine. Consequently [³H]-C₁₆-urea-Cer labeled in the sphingosine backbone was synthesized. Should mt-CDase hydrolyze the amide bond at position “a”, then radioactive sphingosine would be released. No [³H]-sphingosine was detected after TLC separation, indicating that ceramidase does not hydrolyze the “a” amide bond of urea ceramide.

On the other hand, if hydrolysis occurs at position “b” of urea ceramide it will yield N-alkyl amine, which at the expected released levels was not detectable by HPLC. To further eliminate that this potential product may be an inhibitor, we examined the effect of N-alkyl amines: C₈ (octyl), C₁₂ (dodecyl), and C₁₈ (stearyl) on mt-CDase activity. All tested amines at 0-1.88 mole % (0-150 μM) inhibited the release of [³H]-palmitic acid from D-erythro-C₁₆-ceramide (0.628 mole %, 50 μM). N-octylamine was the least potent inhibitor, showing a maximal inhibition of 40% over a wide range of concentrations [0.25-1.88 mole % (20-150 μM)]. On the other hand, N-stearylamine (C₁₈) exhibited the highest inhibitory effect (54%) on mt-CDase (FIG. 7c) but was significantly less than that obtained by the urea isoster (85%), thus ruling out the possibility of bond hydrolysis at the “b” amide bond.

These results indicate that urea ceramide is not a substrate but is a competitive inhibitor of the enzyme.

### 6.3 Results: Anti-Cancer Activity of Ceramidase Inhibitors

### 6.3.1 MCF7 and Urea-C6-Ceramide

The anti-cancer effect and the effect on cell proliferation of D-erytho-C6-urea-ceramide were tested with MCF7 cell line as described above. Inhibition was observed at concentration of Urea-C6-ceramide as low as a few μM. The IC₅₀ value is 10 μM. Such results indicate that urea-C6-ceramide is a potent inhibitor of cell growth and proliferation and has potent anti-cancer effect.

### 6.3.2 MCF7 and Urea-C₁₆-Ceramide

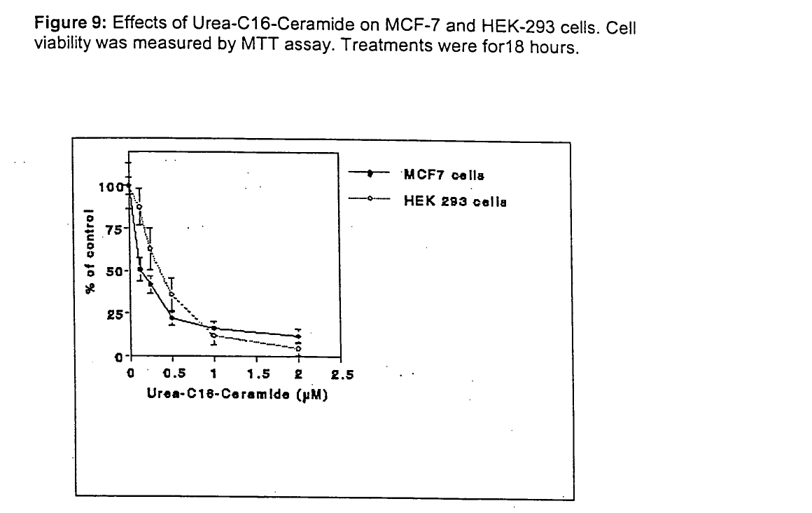

The anti-cancer effect and the effect on cell proliferation of D-erytho-C16-urea-ceramide were tested with MCF7 cell line as described above. Results are depicted in FIG. 9. Such results indicate that urea-C16-ceramide is a potent inhibitor of cell growth and proliferation and has potent anti-cancer effect.

### 6.3.3 HEK-293 and Urea-C16-Ceramide

The effect of D-erytho-C16-urea-ceramide on cell proliferation were tested with HEK-293 cell line as described above. Results are depicted in FIG. 9. Such results indicate that urea-C16-ceramide is a potent inhibitor of cell growth and proliferation.

The present invention is not to be limited in scope by the microorganism deposited or the specific embodiments described herein. The specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

1. A compound having the formula: 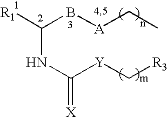
wherein
A is —CH₂CH₂—, trans —CH═CH—, or —C≡C—;
B is —CH₂—, —CH(OH)—, —CH(alkyl)—, —C(═O)—, —C(═NOH)—, or —C(═N—NH2)—;
X is O, S, or NH;
Y is NH, O, C═O, CHR₄, CH₂C═O, or CH₂CHR₄;
R₁ is CH₃, CH₂OH, CH₂SH, CH₂—NH₂, CH₂N₃, CH₂—NH—OH, CH═N—OH, CH═N—NH₂, C(═O)H, C(═O)CH₃, C(═O)CF₃, C(═O)NH₂, CH₂R₅, C(OH)R₅, or C(═O)R₅;
R₃ is H, CH₃, OH, NH2, NH₂.HCl, NHC(═O)NH(CH₂)_{L}N(CH₃)₂, NHCONH(CH₂)_{L}N(CH₃)₂.HCl or C(═O)R₅;
R₄ is H, OH, OR₅, or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle; R₆-phenyl; or R₆-condensed aromatic ring system;
R₆ is H, Cl, OH, CH₃, NO₂, NH₂, N(CH₃)₂, or NHC(═O)(CH₂)ₖCH₃;
L is an integer from 2 to10;
k is an integer from 5 to16;
n is an integer from 4 to 20;
m is an integer from 0 to 26;
with the proviso that the compound is not
(i) (2S, 3R, 4E), (2S, 3R, 4E), (2R, 3R, 4E), or (2R, 3R, 4E) ceramide wherein R₁ is CH₂OH; B is —CH(OH)—; X is O; Y is CHR₄; A is trans —CH═CH—; R₃ is CH₃, H, or OH; R₄ is H or OH; n is 12; and m is an integer from 0 to 24; and
(ii) (2S, 3R, 4E) 3-keto ceramide wherein R₁ is CH₂OH; B is —C(═O)—; X is O; Y is CHR₄; A is trans —CH═CH—; R₃ is CH₃ or OH; R₄ is H or OH; n is 12; and m is 13.

2. A compound having the formula: 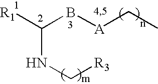
wherein
A is —CH₂CH₂—, trans —CH═CH—, or —C≡C—;
B is —CH₂—, —CH(OH, —CH(alkyl)—, —C(═O)—, —C(═NOH)—, or —C(═N—NH₂)—;
R₁ is CH₃, CH₂OH, CH₂SH, CH₂—NH₂, CH₂N₃, CH₂—NH—OH, CH═N—OH, CH═N—NH₂, C(═O)H, C(═O)CH₃, C(═O)CF₃, C(═O)NH₂, CH₂R₅, C(OH)R₅, or C(═O)R₅;
R₃ is H, CH₃, OH, NH2, NH₂.HCl, NHC(═O)NH(CH₂)_{L}N(CH₃)₂, NHCONH(CH₂)_{L}N(CH₃)₂.HCl or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle; R6-phenyl; or Rr-condensed aromatic ring system;
R₆ is H, Cl, OH, CH₃, NO₂, NH₂, N(CH₃)₂, or NHC(═O)(CH₂)ₖCH₃;
L is an integer from 2 to10;
k is an integer from 5 to16;
n is an integer from 4 to 20;
m is an integer from 0 to 26;
with the proviso that the compound is not
(i) (2S, 3R, 4E), (2S, 3R, 4E), (2R, 3R, 4E), or (2R, 3R, 4E) sphingosine wherein R₁ is CH₂OH; B is —CH(OH)—; A is trans —CH═CH—; R₃ is H; n is 12; and m is 0;
(ii) (2S, 3R, 4E)-N-methyl-sphingosine wherein R₁ is CH₂OH; B is —CH(OH)—; A is trans —CH═CH—; R₃ is CH₃; n is 12; and m is 0; and
(iii) (2S, 3R, 4E) 1-O-methyl-sphinogosine wherein R₁ is CH₂OH; B is —CH(OH)—; A is trans —CH═CH—; R₃ is H; n is 12; and m is 0.

3. A compound selected from the group consisting of D-erythro-C16-urea-ceramide, cis-D-erythro-sphingosine, D-erythro-dihydrosphingosine, 1-O-methyl-D-erythro-sphingosine, 3-keto-sphingosine, 3-keto-dihydrosphingosine, 3-keto-dehydrosphingosine, N-stearyl-D-erythro-sphingosine, N,N-dimethyl-sphingosine, C₁₆-urea-ceramide, and C₆-urea-ceramide.

4-12. (Canceled)

13. A compound having the formula of 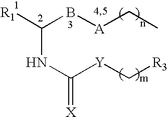
wherein
A is —CH₂CH₂—, trans —CH═CH—, or —C≡C—;
B is —CH₂—, —CH(OH)—, —CH(alkyl)—, —C(═O)—, —C(═NOH)—, or —C(═N—NH₂)—;
X is O, S, or NH;
Y is NH, O, C═O, CHR₄, CH₂C═O, or CH₂CHR₄;
R₁ is a phosphate group;
R₃ is H, CH₃, OH, NH₂, NH₂.HCl, NHC(═O)NH(CH₂)_{L}N(CH₃)₂, NHCONH(CH₂)_{L}N(CH₃)₂.HCl or C(═O)R₅;
R₄ is H, OH, OR₅, or C(═O)R₅;
R₅ is a five-membered monocyclic heterocycle; six-membered monocyclic heterocycle; five- and five-membered bicyclic heterocycle; six- and six-membered bicyclic heterocycle; five- and six-membered bicyclic heterocycle; five-, five-, and five-membered tricylic heterocycle; six-, six-, and six-membered tricylic heterocycle; five-, five-, and six-membered tricylic heterocycle; five-, six-, and six-membered tricylic heterocycle; six-, five-, and six-membered tricylic heterocycle; five-, six-, and five-membered tricylic heterocycle; R6-phenyl; or R6-condensed aromatic ring system;
R₆ is H, Cl, OH, CH₃, NO₂, NH₂, N(CH₃)₂, or NHC(═O)(CH₂)ₖCH₃;
L is an integer from 2 to10;
k is an integer from 5 to16;
n is an integer from 4 to 20; and
m is an integer from 0 to 26;
with the proviso that the compound is not (2S, 3R, 4E) ceramide wherein R₁ is CH₂OH; B is —CH(OH)—; X is O; Y is CHR₄; A is trans —CH═CH—; R₃ is CH₃, H, or OH; R₄ is H or OH; n is an integer from 12 to16; and m is an integer from 0 to 24.

14-16. (Canceled)

17. A method for increasing mitochondrial ceramidase activity comprising contacting a composition comprising mitochondrial ceramidase activity with the compound of claim 13.

18. (Canceled)

19. A method for treating or preventing a disorder in a subject characterized by deficient cell proliferation or growth, or in which cell proliferation is desired, said method comprising administering the compound of claim 13 to the subject.

20. (Canceled)

21. A pharmaceutical composition comprising a pharmaceutically acceptable carrier and the compound of claim 13.

22-36. (Canceled)

37. A method for increasing mitochondrial ceramidase activity comprising contacting a composition comprising mitochondrial ceramidase activity with the compound of claim 1.

38. A method for treating or preventing a disorder in a subject characterized by deficient cell proliferation or growth, or in which cell proliferation is desired, said method comprising administering the compound of claim 1 to the subject.

39. A pharmaceutical composition comprising a pharmaceutically acceptable carrier and the compound of claim 1.

40. A method for increasing mitochondrial ceramidase activity comprising contacting a composition comprising mitochondrial ceramidase activity with the compound of claim 2.

41. A method for treating or preventing a disorder in a subject characterized by deficient cell proliferation or growth, or in which cell proliferation is desired, said method comprising administering the compound of claim 2 to the subject.

42. A pharmaceutical composition comprising a pharmaceutically acceptable carrier and the compound of claim 2.

43. A method for increasing mitochondrial ceramidase activity comprising contacting a composition comprising mitochondrial ceramidase activity with the compound of claim 3.

44. A method for treating or preventing a disorder in a subject characterized by deficient cell proliferation or growth, or in which cell proliferation is desired, said method comprising administering the compound of claim 3 to the subject.

45. A pharmaceutical composition comprising a pharmaceutically acceptable carrier and the compound of claim 3.
